SEQUENCE LISTING (17 sequences)

SEQ ID NO: 1 - DNA, 1884 bp, Clostridium perfringens
  CDS (631)..(1884): SAM gene
cataaaaaatcactccttttatatttttaaaaacattatcacttttttattaattattttcaatataattagtataactggtcaaaaattatacataattggactgcagcacttatcttatatttaataccaagtttatcttcttctattatattaaaaatattttctatataattatatttattatttacttaagtatttagtaaatgaattactatacttctcttcatgattaagctttatttaaatttttatatatattaattaactaaatttaatactttattttactaaaaaaattatatttgtatttaaataaaaaactactattttttagtaaaataaagtatttataaaaatattaatatacatttagtttatttttgatatatcaaaaacttttttatttttaaaaaaagttgataatttaatatagttgaattttgctataatatactaaagtttaaataaggagttgataaacatgaaatttaaagttttagaaaaaaactttttaactatgtgtgagccaaatttttgttgttcaccagtaacaaatttataaaatattatactaaaaaaactaactctgatttcatcagggttagttttctatatggaggttttaaaatgaaaaaatcaatgtataatatttatatacaaggaaaaaaagaaaccgcaattttcaacctattaacaagaaatataattgttatttcaaacaatgaatatctaaattttgataaattaataaatgatgaagaatgtaaagaacaaaaatctatgggtttttctttagaaaataatattaatgaaattgaattaatgaaatatactttaaataaaggtaaattttccgaaaaatcaatgacattatttttatcactcacaagacaatgtaatttaaattgcatttattgctatcaagatagacgaaaaacatttgatgaaggaaataattttttaaataaaactgcttggttaaaaatttttgaatttttaaagaaaaaatctattaatttaaatgaattacatataactttatttggtggagaacctttactaaataaatcaattattttacaaattattgatgatttaaattctttaaaaaatcaaactctaaaaatatatataaatttaataactaatggagttcttttagataaaaatttttgcgaaaaaatatataaatctataaattatattcaaattacaatagatggtaataaatcaactcataatcaattaagagtttttccagatggttctggaagctttgatattatttataataatttaaaggaagtaattaatctatttgaaaataaaatagaattaagaattaatgtaaataaagaaataatattaaattgcttttctttatttaaaaaattaaaggaagattctttaaataaaagtatagccataaatttacatccaatttttgaaaatcaaagctctttcgaatatggctgtaaaaagaatacagacaaagaccttatattacaaattaatgatttatataattatttatctaaacataattttaaatttagaaaaaacttcatagagggtccatgtatagctaaacatataaatagttttgctatagatgaaaatctaaatatttataaatgtccaggctttttatattctaagtctaatggatttataaatgatattggagaaataaactttttagactctgatttctttaaagaagtaaattctgaacaaaaatgtattaattcttgtatatatgctcctatttgttatggtggatgtacttggcaaaataaatgtaataaagagattttagatttaactttagaatcacaattaaaatcttatttaatatctagatataattttgaaatataa

SEQ ID NO: 2 - protein, 417 aa, Clostridium perfringens
MKKSMYNIYIQGKKETAIFNLLTRNIIVISNNEYLNFDKLINDEECKEQKSMGFSLENNINEIELMKYTLNKGKFSEKSMTLFLSLTRQCNLNCIYCYQDRRKTFDEGNNFLNKTAWLKIFEFLKKKSINLNELHITLFGGEPLLNKSIILQIIDDLNSLKNQTLKIYINLITNGVLLDKNFCEKIYKSINYIQITIDGNKSTHNQLRVFPDGSGSFDIIYNNLKEVINLFENKIELRINVNKEIILNCFSLFKKLKEDSLNKSIAINLHPIFENQSSFEYGCKKNTDKDLILQINDLYNYLSKHNFKFRKNFIEGPCIAKHINSFAIDENLNIYKCPGFLYSKSNGFINDIGEINFLDSDFFKEVNSEQKCINSCIYAPICYGGCTWQNKCNKEILDLTLESQLKSYLISRYNFEI

SEQ ID NO: 3 - DNA, 1679 bp, Clostridium perfringens
ttaaaaataggttctaacaaccatttctgtaaacataccagtaggattttttctttggcttaaaaagtattcctttttatctttaatcttttcttttgaccatctcatataatcactctcctaaaatattatggttattataataaaaagaaagtatctaaaatagatactttcttagttataagttgaaatgtgaaaagctataattatgatataaaaactcttaaaagatacaccttaattataactttaaatttaataaataaacagtatattactctttatttttcttaaaaaaatttagtattgcatttttatcttttctacgtggaaatgttaaatctaagaactccatcattttctttttagtttgggttcatcctagtattgaagtattctttagaatatttcctctctttttgaaaatttcatcttcagtatctctaaatcttgctttttctgcagcatttattatacccgcagccatttttatattatcctggatttgtttctcgtttaatgaaccgttaaaattcctaaattcaactgtaggagctctatctcttgctatattagttaagttaataccataatatataatttccttcagaaatttttgctgccctccttcttacatcctcatcattatccatgttgaatcggatatatttactttcatcggccctaggactaaatggagtggcataatgtctggcattacttcttactcttcctaaatctccacctgcagctctataaatacaatcctcatatacttctatagtcttaaaaaatcttctccatctttgtcttgcagtatctagtttatccatgtttatatgaacatggccaccgcacctttgatctactctagccccatgtcttttagcaacatcacatattatttttatttgttcccacgtttctggagtatctttaagaataggggaaactatttctccgctggcatctccatcggatacggacccatctctctcgagtttccacttacctggctcacttcttgaatgataacttaatcttctaggagagcttactatacctagctcataaagttctatggctatagcattaacatcaccaccaacaaactcaagttcaactccgaatgtactattgtttccgtttagtacgttttcgtactcatattctatagaatcgtcattaatttctgcaaaatcgttattgaaatcagcttcattgtctgtataaaagtgattatcgtcttgttcttccccacgaatatttctatttatttcatctcttatatttctagctcttctagtagcttctacattgtttatagtatctacttctgcaacttcgcctaaagcttttctgaccgctattaagtgtctgcagcattcatttctataaacatatttgccacattcacagctattttcattcatatcaacaatgtaagttccttcttcagttgaacttacttcatatcgattattacccaaggaaactatatttatatcttcaattctgtttctggttctagataacccttgaacagttacaatgttatctaatctaccattaacagcattaactaaatcttctattattccttcagaaagattagatcctgagccatcaattaatccttccgttgtatttgcaatagtattgcttcttcttttaggcat

SEQ ID NO: 4 - DNA, 268 bp, Clostridium perfringens
gccgtgctagatggggagttagcggtgccctgtaacctgcaatccgctacagcaggatcgaagtcctcatagaggctctaacttgtggagtctgccctatgtaagtggtgttgagagttgggccccacgcaatggaaacctgtgaacctcgtcaggtccggaaggaagcagcgataagcagtcattttcatgtgccgtggatacgtctggcttgagctaactgcataggtaacgttcataagttattgtcgaagtgaggtgcacggtt

SEQ ID NO: 5 - DNA, 3315 bp, Clostridium perfringens
  CDS (1)..(3315): ColA gene
atgaagaaaaacttaaaaaggggagagctaacaaaactgaagttagttgaaagatggtcagctacctttactttagcagcatttattttatttaatagctcatttaaagtatttgcagctgataaaaaggtagagaatagtaataatggacagattactagagagattaatgctgatcagatttctaaaacagaattaaataatgaggtagctacagacaataatagaccattaggacctagtattgctccatcaagagcaagaaacaacaagatctatacattcgatgaacttaacagaatgaattatagtgatctagttgaattaataaaaacaataagttatgaaaacgtaccagacttatttaattttaatgatggttcatatactttctttagtaatagagatcgtgtacaagctataatatatggtctagaggatagtggaagaacttatacagcagatgatgataagggaattccaactttagttgagtttttaagagctggatattatttaggattttataataaacaattatcatacctaaatacaccacagttaaaaaatgagtgtttaccagctatgaaagcgattcaatataatagtaattttagattaggaacaaaggcgcaagatggagttgttgaggctttaggaagacttataggtaatgcttcagcagatccagaagttattaataattgcatatatgttttaagtgattttaaagataatatagataagtatggttctaactatagcaagggaaatgcagtattcaaccttatgaaaggtattgattattatactaattcagtaatatacaatactaagggatatgatgctaaaaacactgagttctataatagaatagatccatatatggaaagattagaaagtttatgtacaataggtgataagttaaataatgataatgcttggcttgtaaataatgccttatattacacaggtagaatgggtaagtttagagaagacccatcaatatctcaaagagctttagaaagagctatgaaggagtatccttatttatcatatcaatatattgaagctgccaatgatttagatttaaattttggtggcaaaaattcatcaggaaatgatatagatttcaataagataaaagcagatgcaagggaaaaatatcttccaaaaacatatacttttgatgatggtaaatttgtagtaaaagctggtgataaagtaacagaagagaagataaaaagattatattgggcttcaaaggaagttaaggctcaattcatgagagtagttcaaaatgataaggctttagaagagggaaatccagatgatattttaactgttgttatttataactcaccagaagagtataagttaaatcgtataataaatggatttagtactgataatggtggtatatatattgaaaacataggaactttctttacttatgaaagaacaccagaggaaagtatatatacattagaagaattattccgtcatgaatttactcactatcttcaaggtagatatgtagttcctggaatgtgggggcaaggagaattctatcaagagggagttttaacttggtatgaagaaggaacagcagagttctttgcaggttcaactagaactgatggaataaaaccaagaaaatcagttacacaagggttagcttacgatagaaataatagaatgtctttatatggtgtattacatgctaaatatggctcatgggatttctataattatggatttgctctatcaaactacatgtacaacaataacatgggaatgtttaataagatgacaaattacataaagaataatgatgtatctggttataaagattatattgcatcaatgagtagtgattacggattaaatgataaatatcaagactatatggattctttattaaacaatattgataacttagatgttcctttagtttcagatgagtatgtaaatggacatgaagctaaggatataaatgaaataactaatgacataaaagaagtttcaaatataaaagatctttctagtaatgttgaaaagtctcaattctttactacttacgatatgagaggaacatatgtagggggaagaagtcaaggggaagaaaatgactggaaagatatgaattctaagttaaatgatatgttaaaagaattatctaaaaagagctggaatggttataaaactgttactgcatactttgtaaaccataaagtagatgaaaatggtaactatgtttatgatgttgtattccatggaatgaatacagatacaaatactgatgttcatgttaataaagagcctaaggctgttataaaatctgattcttcagtaatagttgaagaagaaattaattttgatggaacagagtcaaaggatgaagatggtgaaatcaaagcttatgaatgggactttggagatggagaaaaatctaatgaggctaaagccactcataagtataataaaactggagaatatgaagtaaaattaacagttacagataataacggtggaataaatactgaaagtaaaaagataaaagtagtagaagataaacctgttgaagttataaatgaaagcgagccaaacaatgattttgaaaaggctaaccaaatagctaaatctaatatgttagttaagggtactttatcagaagaggattattcagataaatattattttgatgtagctaaaaaaggcaatgttaaaatcactcttaataatttaaattcagtaggaataacttggacactttataaagagggagacctaaacaattatgttttatatgcaactggaaatgatggaacagaattaaagggtgaaaagactttagagcctggaagatactacttaagtgtatatacttatgataatcaatcaggagcttacacagtaaatgtaaaaggaaaccttaaaaatgaagttaaagaaacagaaaaggatgctataaaagaagttgaaaataacaatgattttgataaagctatgaaggtagacagtaatagcaaaatagttggaacattaagcaatgatgatcttaaggatatttatagcatagatatacaaaatccaagtgacttaaacatagtagttgaaaacttagataatataaaaatgaactggttattatattcagctgatgatttaagtaactatgtggattacgctaatgcagatggaaataaattaagtaacacttgtaagttaaatccaggtaaatattacttatgtgtttatcaatttgaaaactcaggtactggaaattacacagtaaacttacaaaacaaataa

SEQ ID NO: 6 - protein, 1104 aa, Clostridium perfringens
MKKNLKRGELTKLKLVERWSATFTLAAFILFNSSFKVFAADKKVENSNNGQITREINADQISKTELNNEVATDNNRPLGPSIAPSRARNNKIYTFDELNRMNYSDLVELIKTISYENVPDLFNFNDGSYTFFSNRDRVQAIIYGLEDSGRTYTADDDKGIPTLVEFLRAGYYLGFYNKQLSYLNTPQLKNECLPAMKAIQYNSNFRLGTKAQDGVVEALGRLIGNASADPEVINNCIYVLSDFKDNIDKYGSNYSKGNAVFNLMKGIDYYTNSVIYNTKGYDAKNTEFYNRIDPYMERLESLCTIGDKLNNDNAWLVNNALYYTGRMGKFREDPSISQRALERAMKEYPYLSYQYIEAANDLDLNFGGKNSSGNDIDFNKIKADAREKYLPKTYTFDDGKFVVKAGDKVTEEKIKRLYWASKEVKAQFMRVVQNDKALEEGNPDDILTVVIYNSPEEYKLNRIINGFSTDNGGIYIENIGTFFTYERTPEESIYTLEELFRHEFTHYLQGRYVVPGMWGQGEFYQEGVLTWYEEGTAEFFAGSTRTDGIKPRKSVTQGLAYDRNNRMSLYGVLHAKYGSWDFYNYGFALSNYMYNNNMGMFNKMTNYIKNNDVSGYKDYIASMSSDYGLNDKYQDYMDSLLNNIDNLDVPLVSDEYVNGHEAKDINEITNDIKEVSNIKDLSSNVEKSQFFTTYDMRGTYVGGRSQGEENDWKDMNSKLNDMLKELSKKSWNGYKTVTAYFVNHKVDENGNYVYDVVFHGMNTDTNTDVHVNKEPKAVIKSDSSVIVEEEINFDGTESKDEDGEIKAYEWDFGDGEKSNEAKATHKYNKTGEYEVKLTVTDNNGGINTESKKIKVVEDKPVEVINESEPNNDFEKANQIAKSNMLVKGTLSEEDYSDKYYFDVAKKGNVKITLNNLNSVGITWTLYKEGDLNNYVLYATGNDGTELKGEKTLEPGRYYLSVYTYDNQSGAYTVNVKGNLKNEVKETEKDAIKEVENNNDFDKAMKVDSNSKIVGTLSNDDLKDIYSIDIQNPSDLNIVVENLDNIKMNWLLYSADDLSNYVDYANADGNKLSNTCKLNPGKYYLCVYQFENSGTGNYTVNLQNK

SEQ ID NO: 7 - DNA, 1001 bp, Clostridium perfringens
tcaaaatagagtgcttttacttataaaacttaatatatttattcctaaaatatcagataaaattaagactatgttatatataatgagaaggaaattaaatatttttagtattttatttattatcttttcagtttttgatccagttttaattttggctattttaaagttagttttccaaggatagaaaagttctattccttctgaagttaaaaaatctaaaactaagtgggatatatatccagctataaatcctattgaaattattaataatattatgttgaagttactaacatttaaaataatatataaggttaatactattaaagatagacatagtaaactatgagtaaagccacgatgtctacatttactacctatatgctttgaaagaaaaggatacatttgactaattgaagattttctttgatctatatcaggaaatagagatcctatataagaaaaatgaaaaaataatgctattaataaaattttataagctatatcatatggaaataagaacaggcttaaaattgtatttaacattattaaagaaatgcatacgcctccagttgaatgagtttcttttgtcataaaatctcctttaaaaataattatcattagtttatattaaaaatattatcataatattcattttacataaagaagaaaaaaataaaaatcctaaaaatagagtggacagttctatttctaggatcaatagttatggtggtaattttatgtaaggttttttactaaattaaagtaaaatcattaataagtaattatttatttttaattacaattatggaatatgcaattgaaaatattgatattaaagcaagtatgggacttataatatttgaaacaacttctaaagatgtaaaataagatactctttgttcaatatataaagctatgctatttttaagtattaagggagaaaaaagagttattaatgtaattaaaattgcacttgttaaaatcctttttgtatgtttcaa

SEQ ID NO: 8 - DNA, 960 bp, Clostridium perfringens
  misc_feature (527)..(527): n is a, c, g, t or u
ttacagataatattctattttatgatcttgccaatttattttaaacataaattcgttatattcacttgttgacgaaagtttgtttgttcctcgccattgagtagtttcccaatttaaaatatagtcattatcaaatctttgatattcaactattattacagattctttagcatttttaggtgctgttaatgctaaagccatattgggtgaaaatccaccagaaattaatgttgataaatctctatcatctgtgaaatttttatcaccattattatacaatcttgatttcatgaataattgatttccataaatagcatgataagaatctatattataaccgtccttagtctcaacaaattttatatcccatgatgctaaatttgcatcatcttttctttgaattgttctaaaatcaggttgttcatagcttatagtattttggacattatatgaagcatttattccagcaccagcagtttttccttcaacagatatattaccgcctatagaataaccaattgaattanatacatcttttttatctatagttttaggaatagaatttgctatattattatttacatcagcactttttacattaattctataagtttcaggccatttcatttttccgtaatatttagaaccaaaaatctgtttatcagaaggtataaatccttctaaatttaataaagcagttttcttatcagaatgaggatcttcaataaatgttccacttaaagttgccttccaacctttatgtgaagcggtatcactagaagtatatttaatagcttcctttccattttcttttattatttctccacttagatttttcaactcaattttgtttatgtcatttaattcacttgcaaaaacttgagtttgagttgaaaaaaggcttgtactaattacacttgtaagaactagtgtaattgaaataatttttaatcttttcaa

SEQ ID NO: 9 - DNA, 562 bp, Clostridium perfringens
tgtttatcatcccttttctctcatttttatatactcataaatatatcataaaaagcaaaatctatccataatatatttaaaaaatgagtatatagtttttaataaaattatatttaattatgtataaaaatcataagttaaattgctctatataatatttccaaataagattaattttatgaaatagaaatgaaaaaagttctgcaaagtgcagaacttttaatttatgaataaacttgcgttaattatcaattatctattcattttttgagcttttcttgcttttctgcaagctgggcatctaactggatcgttttcaaatcctttttctttgaagaattcttgttctcctacagtgaatacgaattcttttccacagtctttacaaactaaatttttatcttccatttcttttacctcctgtaaaatttactaggtacgtttgagatctatactataaaactaataaaataaattttacaggacagaaataaagtacatatctactaaattttattttatatattgattatatcatatagaaaaaagaatacaatacttt

SEQ ID NO: 10 - RNA, 84 nt, Gallus gallus
gucugucauacucuagcagcacguaaauauugguguuaaaacuguaaauaucuccaguauuaacugugcugcugaaguaaggcu

SEQ ID NO: 11 - RNA, 68 nt, Gallus gallus
cuccggugccuacugagcugauaucaguucugauuuuacauacuggcucaguucagcaggaacaggag

SEQ ID NO: 12 - RNA, 63 nt, Gallus gallus
uguuaaugcuaaucgugauagggguuuuuaccucugaaugacuccuacauguuagcauuaaca

SEQ ID NO: 13 - RNA, 76 nt, Gallus gallus
gagaugacaugcuucuuuauauccccauauggauuaggcugcuauggaauguaaggaagugugugguuucagggag

SEQ ID NO: 14 - protein, 77 aa, Clostridium perfringens
MKEIIYYGINLTNIARDRAPTVEFRNFNGSLNEKQIQDNIKMAAGIINAAEKARFRDTEDEIFKKRGNILKNTSILG

SEQ ID NO: 15 - protein, 196 aa, Clostridium perfringens
MTKETHSTGGVCISLIMLNTILSLFLFPYDIAYKILLIALFFHFSYIGSLFPDIDQRKSSISQMYPFLSKHIGSKCRHRGFTHSLLCLSLIVLTLYIILNVSNFNIILLIISIGFIAGYISHLVLDFLTSEGIELFYPWKTNFKIAKIKTGSKTEKIINKILKIFNFLLIIYNIVLILSDILGINILSFISKSTLF

SEQ ID NO: 16 - DNA, 5628 bp, Clostridium perfringens
  CDS (673)..(5628): TpeL gene
gatcttaatgaacctaatacagatatatttgtttatgatttaatagacaagctttcagaaaaagaaagaaaaataattaaatataaatacatacatggaaaaagtgatgttgaaatagggaaattacttaattattctagacagcacgtaaaccaaataaaaaatagagcattaaaaaatttgaagaaatttttagaggagtgatttaatctaaatattaaaattaaatatttataggaggttaattgtggattcagaactgtttaaaattatggctactcaaggagcttttgctttactattttcctacttgttattttatgtattaaaagaaaatagcaaaagagaagaaaattatcaaaatataattaaggagcttacagaattactaccaattataaaaagtgatgttgaagacataaaaaataaattgaataataattagaaaaaatttataaaaaatataaattttaaatttttatttacaaaatttaaaattagaactctttatataatgtataagaaatttaattctaagtatataaaatactaaaggagggttaagaagaataaaatttgaaaggaaaaaatttgtgcaaattcaaatttataattagattgaagggaggatttgatgtaaatataaattaagttttacatcaacaataatatatggggttaatgtcaaaagaacaattaattatattagcaaaaaattcaagtccaaaagaaggagaatataaaaaaattttagaattactagatgaatataatttattaaataattctgttgaaaaaaatagtatagatttatatttaaaacttaatgaattatctaaatcaattgatatttatttaaaaaaatataagaattcaaagagaaataatgcattatatcaattaaaatctgatttaacaaaggaagttattgaaataaaagatactaatttaaaacctttggaaaaaaatattcattttgtttgggttggaggaatgataaataatatatctattgattatataaatcaatggaaagatattaatagcgattatgaaactattatatggtatgatagtgaagctttgttagtaaatatattaaaaaaagctataatagactcatcaaataaagaagttttaactaaatatgaatcagtattaaatgataatagctttgattcaaataaattttatagagaaagaatggaagttatttttagaaagcaaaaagagtttaataattattataatacaaatgataactatactaaatctttaaatgatgtaattaaagtttatttaatagaaaaatatttaaaaactgatgaagaattagaaaaatatattaatgaaagtaaagaagtttttaaggctaatggagctaaagatataagagaatacgatatactagatgatgttgaattaaaatcaatatatgagcaagagttattaatgagatttaatttagcatctgcatcagatattattagagtgattgtattaaataaattaggtgggatttatttagatgtagatgtgcttcctggaataaaaaaacatatttttaaagatattaataaacctacaaatatttctgaaaataaatggcaaatgattcaattagaaactattatgaaatataaacaatatataaaaggatatacagaaaattcatttaaaaatttaccaagtgatttgcaggaaatgttacaagaaaaagttgttgaaaaaaatcttaaaagtgatatatttcagagattaggggatatatttatttcagaattagatacaaaaatagcttttatgtttgggaaaattgctaatcaagtattaatatctaaaaaaaattcatactcattaaatttaataattaatcaaatcaagaatagatataatattattaataaatgtttatcttcggctattgaaaaaggatcaaattttaataatacagttgatatattcattcaacaattaaatgaattctatgttaatgaaggcttttttgttagtaaagtaatgggatatttaggtgatggatatatgcctgatatgagagcgacacttaatataagtggtccaggaatatatacagctgcttattatgatttattatattttaatgagaggtcattaaatcctcaaattttacaagaagatttgaaatattttgaagttccacaagctctaatatcacaacaaacagaacaagaaataaatagttcatggacatttaatcaagttaaatcacaaatagaatataaaaagctagtagaaaaatatactaataaatcattaagtgaaaatgataaactaaattttaatgagaataaaatcattgataaagtagaattactaaatcgaattaatagtaataatttaattaattttgatgataaagaatacttaagatatataattcaattacaaggtgataaagtaagttatgaggctgcaataaatttatttattaaaaatccttcaaattcaatactagttcaagaaattaataatataagctactattttaattcagaatataaatcaatagattctatacaatttgataatattcctgaaatattaaaaggtaaaaataaaattaaattaacatttataggtcatggagaagaagaatttaatacagagagatttgcttcattaacagtaaaggagttttcaaaaaaaatatataaagttttagatatgataaagagcaatacgaatgttaaagaaattcaaatagatttgttaggatgtaatatgttttcatataatataaatgtagaagaaacatatcctgggaaattattaaaagtagtattagattatgtagataaaatttataatgcagatattaaaccagaaataaaaataagtgccaatcaatatgaagtcagaataaataaagatgggaaaaaagaattactatctcattcaggagagtggctttctaaagaagaagctataataaaagatattgcgagtaaagaaattatttattttgatacaagagaaaatactattaaagcagaagctaaaaatattatggagttgataacttttagaaatagtctagataaaaaattaaatgatttagaaagtttaattaataataattttattgtaaatgaagtatatgaacaattaacatcaaataatttatatagaacaagttttagaaattcttttttagatactataaattttatagaagatgtttctcaagagttatatgaaataaagataaaaaataatataaacgatgattatatcatggcattagatgaaataaagcaagataataatatttcagaaattaaatttattaatattaattcaggagaatttagggttgtaagagaaaatagtaggttaattaataaatttaaagaagattttaaatttatgattaataatattaagaggttgataaaatttagtgataataacaagctaataacatcatttgaattaaagaatatagagtcaagcagtggagtaagtacattaaatacaagttttttaattcaatctatgattgattataaagcgcaaaactttgacttcaataaattaagtacatcagtaaaagtacagatttattgtcaaataactaatataagtttatcagaaattcaagatgctagtaatttagtaaaaattatagctgaagcaaatgaaattgaaataaatttaataccaacattagcaaatgctataccattaataacaacaatagttgatggaataaatttgattgcaaatatagatgaattaataaatactaaagatgaattactaagaaaggaattagcagctagaattggaattatttcatctaatatgacagcagctattagttcttatattttatattttactgaatttggagaagtatttaatcctttattagtgccaattgcaggtatatcaagtggtattcctactttagtaaataatattttaattttagaggaaaaatctaaagaaataacagaatatttttcacatataagtaaagtagaaagtgatggattatttaaagtgtctgataataacagtatattgattccacttgatgatattgcaatttctaagattgattttaataaaagagaagttatcttagataaattagatatatgggcaatggaaggaggatcaggattaagtggtaaagaaacttttttttctgctccgtatataaatgaaaatcttcctaaattatctattgataatttgcttaagatagatataaataaaatagatttttcaattaaaggtatgatgctacctaatggcgttagtaaaacattaggttatgagttcaatacagttgccaatatatatgaattagaaaatgatggtgtaaatttattaaatagaattagagataattatccaggacagttttattggagatattatgctactctatttaattatggtatggtaaatttaaaaattaattatcatgacacagatgtaaaaataaatttagataatgttgatcgtatgtttatagtaccgacattaacaatagatcaagctagagaaaaactttcatataattttaatggagcaggagctaattattatatatacctttcgtctcaaccaataaaaatatttattaatgggactaaagaagataattggattcttaatatagatgatattgttaaagaagttacaattgttaataatagtatagtaagaggaaagtttattgaaaatatatttaaaaaattaactattgaggataataaaataattataggaaatcaaaaaatatatttaaaaaataacaaaacaaatatacatttttctgtatctattttagatggagtgaatttaatggtagaagtagactttaataataaaatgtatcatttatcacttcaaggaaacgaaaaaacaatatgtaataatatgaatattatacaaaataacataaataaattatttggctcttcaaatataaaatcaataccatatttctataaaaataatggattagagaattgtattggagtattctcaataaaaaacaatgaatttatttatatgttaaaatatgatgataaaaataaaatatataaatataaagataataatttagtatgtggttttaatttatttgatatatcttatgtagatataatttctaatggagataagaattatttgaaaggaatttggaatacatcagttaataattcagaatataaaattgaatttttagttgaattaatagaccaaaatactattgaaatcattaaattgaatttaagtgatggattaataaaatcatttttaactatattagataatttagaaagtttagatgtaaaaatttcaacaatatatgattttttaaaaaatattctttttagggatgtatttttagaaaatgagtcattaaattttattgtaagtaaaggctttgttttgacagggaataattcaaaaaataaatacgagtttatatgtaatagtgaaaatatacaattatatattactgaactttacactaatggtactaaatttaggaattggcttgacaatggtaagatattagtatcatcaacaggtgaaagttcaaataatttaattgacatataa

SEQ ID NO: 17 - protein, 1651 aa, Clostridium perfringens
MGLMSKEQLIILAKNSSPKEGEYKKILELLDEYNLLNNSVEKNSIDLYLKLNELSKSIDIYLKKYKNSKRNNALYQLKSDLTKEVIEIKDTNLKPLEKNIHFVWVGGMINNISIDYINQWKDINSDYETIIWYDSEALLVNILKKAIIDSSNKEVLTKYESVLNDNSFDSNKFYRERMEVIFRKQKEFNNYYNTNDNYTKSLNDVIKVYLIEKYLKTDEELEKYINESKEVFKANGAKDIREYDILDDVELKSIYEQELLMRFNLASASDIIRVIVLNKLGGIYLDVDVLPGIKKHIFKDINKPTNISENKWQMIQLETIMKYKQYIKGYTENSFKNLPSDLQEMLQEKVVEKNLKSDIFQRLGDIFISELDTKIAFMFGKIANQVLISKKNSYSLNLIINQIKNRYNIINKCLSSAIEKGSNFNNTVDIFIQQLNEFYVNEGFFVSKVMGYLGDGYMPDMRATLNISGPGIYTAAYYDLLYFNERSLNPQILQEDLKYFEVPQALISQQTEQEINSSWTFNQVKSQIEYKKLVEKYTNKSLSENDKLNFNENKIIDKVELLNRINSNNLINFDDKEYLRYIIQLQGDKVSYEAAINLFIKNPSNSILVQEINNISYYFNSEYKSIDSIQFDNIPEILKGKNKIKLTFIGHGEEEFNTERFASLTVKEFSKKIYKVLDMIKSNTNVKEIQIDLLGCNMFSYNINVEETYPGKLLKVVLDYVDKIYNADIKPEIKISANQYEVRINKDGKKELLSHSGEWLSKEEAIIKDIASKEIIYFDTRENTIKAEAKNIMELITFRNSLDKKLNDLESLINNNFIVNEVYEQLTSNNLYRTSFRNSFLDTINFIEDVSQELYEIKIKNNINDDYIMALDEIKQDNNISEIKFININSGEFRVVRENSRLINKFKEDFKFMINNIKRLIKFSDNNKLITSFELKNIESSSGVSTLNTSFLIQSMIDYKAQNFDFNKLSTSVKVQIYCQITNISLSEIQDASNLVKIIAEANEIEINLIPTLANAIPLITTIVDGINLIANIDELINTKDELLRKELAARIGIISSNMTAAISSYILYFTEFGEVFNPLLVPIAGISSGIPTLVNNILILEEKSKEITEYFSHISKVESDGLFKVSDNNSILIPLDDIAISKIDFNKREVILDKLDIWAMEGGSGLSGKETFFSAPYINENLPKLSIDNLLKIDINKIDFSIKGMMLPNGVSKTLGYEFNTVANIYELENDGVNLLNRIRDNYPGQFYWRYYATLFNYGMVNLKINYHDTDVKINLDNVDRMFIVPTLTIDQAREKLSYNFNGAGANYYIYLSSQPIKIFINGTKEDNWILNIDDIVKEVTIVNNSIVRGKFIENIFKKLTIEDNKIIIGNQKIYLKNNKTNIHFSVSILDGVNLMVEVDFNNKMYHLSLQGNEKTICNNMNIIQNNINKLFGSSNIKSIPYFYKNNGLENCIGVFSIKNNEFIYMLKYDDKNKIYKYKDNNLVCGFNLFDISYVDIISNGDKNYLKGIWNTSVNNSEYKIEFLVELIDQNTIEIIKLNLSDGLIKSFLTILDNLESLDVKISTIYDFLKNILFRDVFLENESLNFIVSKGFVLTGNNSKNKYEFICNSENIQLYITELYTNGTKFRNWLDNGKILVSSTGESSNNLIDI

(19) United States
(12) Patent Application Publication
(10) Pub. No.: US 2018/0312905 A1
(43) Pub. Date: Nov. 1, 2018

(54) METHOD DETECTING AVIAN NECROTIC ENTERITIS

(71) Applicant: Evonik Degussa GmbH, Essen (DE)

(72) Inventors: Emeka-Ignatius IGWE, München (DE); Holger WALLMEIER, Sulzbach/Ts. (DE); Stefan PELZER, Gütersloh (DE); Monika FLÜGEL, Steinhagen (DE); Thomas BEKEL, Halle (Westf.) (DE)

(73) Assignee: Evonik Degussa GmbH, Essen (DE)

(21) Appl. No.: 15/555,531

(22) PCT Filed: Feb. 19, 2016
(86) PCT No.: PCT/EP2016/053502

§ 371 Date: Sep. 4, 2017

Related U.S. Application Data
(60) Provisional application No. 62/129,293, filed on Mar. 6, 2015.

(30) Foreign Application Priority Data
Jun. 29, 2015 (EP) 15174270.7

(51) Int. Cl. C12Q 1/689

(52) U.S. Cl. CPC C12Q 1/689; C12Q 2600/178

(57) ABSTRACT

The current invention relates to a microvesicle based method of detecting avian necrotic enteritis as well as to new markers which have been identified as suitable for detection of avian necrotic enteritis and/or avian infection by Clostridium perfringens.

The current invention relates to a microvesicle based method of detecting avian necrotic enteritis as well as to new markers which have been identified as suitable for detection of avian necrotic enteritis and/or avian infection involving Clostridium perfringens.

Clostridium perfringens is the main causative agent of avian necrotic enteritis (NE), an enteric disease of poultry that was first described in 1961. NE in chickens manifests as an acute or chronic enterotoxemia. The acute disease results in significant levels of mortality due to the development of necrotic lesions in the gut wall, whereas the chronic disease leads to a significant loss of productivity and welfare. It has been estimated that the disease results in damages of several billion US-Dollars per year for the poultry industry.

C. perfringens is commonly found in the gastrointestinal tract of poultry, the occurrence of necrotic enteritis, however, is sporadic. C. perfringens is a Gram-positive, rod-shaped, spore forming, oxygen-tolerant anaerobe. C. perfringes strains are classified into five toxin types (A, B, C, D and E), based on the production of four suspected major toxins (alpha, beta, epsilon and iota). While type A is consistently recovered from intestines of chicken, the other types are less common.

Early studies on NE suggested that the main virulence factor involved in the disease was secreted by the bacteria, which led to the proposal that a phospholipase C enzyme, called alpha-toxin, was the major toxin involved in pathogenesis. But recent studies showed that alpha-toxin seems not to be an essential virulence factor since alpha toxin mutant strains were capable of causing NE, which questions the role of alpha-toxin in the disease in general. In more recent studies, the novel pore forming toxin, netB, has been suggested to play a major key role in the development of this disease.

Apart from the mere association of the virulence factors per se to incidence of NE, there is still a great burden to show the mechanistic link between these factors to the establishment of the disease in birds or farm animals. Moreover, relating subclinical NE infection to any of these virulence factors is extremely difficult to achieve since birds are void of signs to warrant their examination earlier before slaughter. Therefore a need for an early method of detecting NE, and most importantly, the subclinical forms of NE remains imperative.

Traditionally, the discovery and validation of biomarkers for disease requires the use of tissue, either fresh frozen, or more commonly, formalin-fixed, paraffin-embedded. This has been especially true for mRNA based biomarkers, because RNase degradation of free RNA makes translation of tissue based biomarkers into biofluids such as blood or fecal/cecal samples highly problematic. But monitoring of tissue biomarkers is thus, because of its invasive nature, time-consuming and impractical, when large numbers of samples are involved like for farm animals.

Non-invasive methods such as the measurement of blood parameters have been described for the detection of diseases in birds (Chuku et al., 2012; Aade et al., 2012; Saleem, 2012). Gulbeena Saleem (2012) suggests as an alternative approach for the determination of sub-clinical NE the measurement of the levels of acute phase proteins like ceruplasmin, PIT54 and ovotransferrin in response to C. perfringes challenge in blood serum of the chickens. It turned out that only ceruplasmin seemed to be a suitable acute phase protein correlating with the occurrence of sub-clinical NE. Notwithstanding, this can be seen as a problem, since ceruplasmin is not a specific marker for NE but for diseases caused by other bacterial pathogens (collibacillosis in chicken, Piercy, 1979; salmonella infection in commercial layers, Garcia et al., 2009), as well.

However, it was recently discovered that exosomes and other membrane vesicles found in biomaterials contain stable RNA from human donor cells, including mRNA, miRNA and other non-coding RNA with high specificity to disease conditions (Skog et al., 2008; Nilsson et al., 2009; Li et al., 2009; Shao et al., 2012).

Thus the inventors of the current application wondered, if membrane vesicles isolated from bodily fluids or excrements of poultry might be used to determine poultry exhibiting sub-clinical necrotic enteritis. This might be in particular true due to an accumulation of specific host miRNAs showing correlation to the disease.

Surprisingly it was found that microvesicles isolated from avian bodily fluids or avian excrements did exhibit an alteration in the content of detectable RNA in dependence of the presence of necrotic enteritis. Besides an increase of specific host-miRNAs in the microvesicles due to infection by C. perfringens, surprisingly also mRNA sequences of C. perfringens could be detected in the microvesicle fraction, which are therefore classified as disease specific virulence factors and accordingly as suitable biomarkers indicative for necrotic enteritis.

The occurrence of C. perfringens mRNA in the microvesicle fraction might be explained by the fact that the isolation procedure applied does not only lead to an enrichment of microvesicles of poultry, but also to an enrichment of microvesicles of the infectious bacterium. Surprisingly, besides known putative virulence factors like netB, also previously non-suspective RNA sequences could be identified as being correlative to necrotic enteritis.

It was thus a first object of the current invention to provide a fast and reliable, preferably non-invasive, method for determining avian necrotic enterititis, in particular sub-clinical avian necrotic enterititis, preferably in poultry, more preferably in chicken and/or to identify avian subjects which suffer from necrotic enteritis.

It was a further object of the current invention to identify suitable markers, preferably disease-specific markers, which can be employed in such a method.

Thus a first subject of the current invention is a method of detecting avian necrotic enteritis, in particular sub-clinical avian necrotic enteritis, the method comprising isolating microvesicles from an avian sample, in particular from an avian bodily fluid or an avian excrement, and subsequently determining the presence and/or the level of at least one marker indicative for necrotic enteritis in these microvesicles, wherein the presence and/or an increased level of this at least one marker in comparison to a non-infected control is indicative for necrotic enteritis.

The marker indicative for necrotic enteritis according to the invention is preferably selected from the following sequences of Clostridium perfringens and homologues and fragments thereof: the SAM region (SEQ ID NO: 1), the swim zinc finger region (SEQ ID NO: 3), the scRNA (SEQ ID NO: 4), the colA region (SEQ ID NO: 5), the CPE0956 region (SEQ ID NO: 7), the netB region (SEQ ID NO: 8), the virX region (SEQ ID NO: 9) and the TpeL region (SEQ ID NO: 16); as well as from the following avian sequences and homologues and fragments thereof: mir-16 (SEQ ID NO: 10), mir-24 (SEQ ID NO: 11), mir-155 (SEQ ID NO: 12), mir-206 (SEQ ID NO: 13).

Thus a preferred subject of the current invention is a method of detecting avian necrotic enteritis, in particular sub-clinical avian necrotic enteritis, the method comprising isolating microvesicles from an avian sample, in particular from avian bodily fluids or avian excrements, and subsequently determining the presence and/or the level of at least one marker indicative for necrotic enteritis in these microvesicles, wherein the presence and/or an increased level of this at least one marker in comparison to a non-infected control is indicative for necrotic enteritis and wherein the marker is selected from the following group (“List I”):
a) polynucleotides which have a sequence identity of at least 80%, preferably at least 85, 90 or 95%, most preferably 100%, to a polynucleotide which comprises at least 10, preferably at least 15, 20, 25, 40, 60, 100, 150, 180 or 200, consecutive nucleotides of the nucleotide sequence as depicted in SEQ ID NO: 1;
b) polynucleotides which have a sequence identity of at least 80%, preferably at least 85, 90 or 95%, most preferably 100%, to a polynucleotide which comprises at least 10, preferably at least 15, 20, 25, 40, 60, 100, 150, 180 or 200, consecutive nucleotides of the nucleotide sequence as depicted in SEQ ID NO: 3;
c) polynucleotides which have a sequence identity of at least 80%, preferably at least 85, 90 or 95%, most preferably 100%, to a polynucleotide which comprises at least 10, preferably at least 15, 20, 25, 40, 60, 100, 150, 180 or 200, consecutive nucleotides of the nucleotide sequence as depicted in SEQ ID NO: 4;
d) polynucleotides which have a sequence identity of at least 80%, preferably at least 85, 90 or 95%, most preferably 100%, to a polynucleotide which comprises at least 10, preferably at least 15, 20, 25, 40, 60, 100, 150, 180 or 200, consecutive nucleotides of the nucleotide sequence as depicted in SEQ ID NO: 5;
e) polynucleotides which have a sequence identity of at least 80%, preferably at least 85, 90 or 95%, most preferably 100%, to a polynucleotide which comprises at least 10, preferably at least 15, 20, 25, 40, 60, 100, 150, 180 or 200, consecutive nucleotides of the nucleotide sequence as depicted in SEQ ID NO: 7;
f) polynucleotides which have a sequence identity of at least 80%, preferably at least 85, 90 or 95%, most preferably 100%, to a polynucleotide which comprises at least 10, preferably at least 15, 20, 25, 40, 60, 100, 150, 180 or 200, consecutive nucleotides of the nucleotide sequence as depicted in SEQ ID NO: 8;
g) polynucleotides which have a sequence identity of at least 80%, preferably at least 85, 90 or 95%, most preferably 100%, to a polynucleotide which comprises at least 10, preferably at least 15, 20, 25, 40, 60, 100, 150, 180 or 200, consecutive nucleotides of the nucleotide sequence as depicted in SEQ ID NO: 9;
h) polynucleotides which have a sequence identity of at least 80%, preferably at least 85, 90 or 95%, most preferably 100%, to a polynucleotide which comprises at least 10, preferably at least 15, 20, 25, 40, 60, 100, 150, 180 or 200, consecutive nucleotides of the nucleotide sequence as depicted in SEQ ID NO: 16;
i) polynucleotides which have a sequence identity of at least 80%, preferably at least 85, 90 or 95%, most preferably 100%, to a polynucleotide which comprises at least 8 or 10, preferably at least 12, 14, 16, 18, 20, 25, 40, 60 or 80, consecutive nucleotides of the nucleotide sequence as depicted in SEQ ID NO: 10;
j) polynucleotides which have a sequence identity of at least 80%, preferably at least 85, 90 or 95%, most preferably 100%, to a polynucleotide which comprises at least 8 or 10, preferably at least 12, 14, 16, 18, 20, 25, 40 or 60, consecutive nucleotides of the nucleotide sequence as depicted in SEQ ID NO: 11;
k) polynucleotides which have a sequence identity of at least 80%, preferably at least 85, 90 or 95%, most preferably 100%, to a polynucleotide which comprises at least 8 or 10, preferably at least 12, 14, 16, 18, 20, 25, 40 or 60, consecutive nucleotides of the nucleotide sequence as depicted in SEQ ID NO: 12;
l) polynucleotides which have a sequence identity of at least 80%, preferably at least 85, 90 or 95%, most preferably 100%, to a polynucleotide which comprises at least 8 or 10, preferably at least 12, 14, 16, 18, 20, 25, 40 or 60, consecutive nucleotides of the nucleotide sequence as depicted in SEQ ID NO: 13;
m) polynucleotides (DNAs and RNAs) which are complementary to the sequences according to (a) to (l);
n) polynucleotides (DNAs and RNAs) which are complementary to the sequences according to (m);
o) polynucleotides which comprise the polynucleotides according to (a) to (n).

In this method the sequences are preferably selected from the following group (“List II”):
a) polynucleotides which have a sequence identity of at least 80%, preferably at least 85, 90 or 95%, most preferably 100%, to a polynucleotide which comprises at least 10, preferably at least 15, 20, 25, 40, 60, 100, 150, 180 or 200 consecutive nucleotides of the nucleotide sequence according to position 1 to 630, in particular 420 to 630, preferably 460 to 560, of SEQ ID NO: 1,
b) polynucleotides which have a sequence identity of at least 80%, preferably at least 85, 90 or 95%, most preferably 100%, to a polynucleotide which comprises at least 10, preferably at least 15, 20, 25, 40, 60, 100, 150, 180 or 200, consecutive nucleotides of the nucleotide sequence according to position 600 to 1000, in particular 670 to 760, of SEQ ID NO: 3,
c) polynucleotides which have a sequence identity of at least 80%, preferably at least 85, 90 or 95%, most preferably 100%, to a polynucleotide which comprises at least 10, preferably at least 15, 20, 25, 40, 60, 100, 150, 180 or 200, consecutive nucleotides of the nucleotide sequence according to position 60 to 240, in particular 80 to 220, of SEQ ID NO: 4;
d) polynucleotides which have a sequence identity of at least 80%, preferably at least 85, 90 or 95%, most preferably 100%, to a polynucleotide which comprises at least 10, preferably at least 15, 20, 25, 40, 60, 100, 150, 180 or 200, consecutive nucleotides according to position 1300 to 1800, in particular 1400 to 1700, of (the sequence as depicted in) SEQ ID NO: 5;
e) polynucleotides which have a sequence identity of at least 80%, preferably at least 85, 90 or 95%, most preferably 100%, to a polynucleotide which comprises at least 10, preferably at least 15, 20, 25, 40, 60, 100, 150, 180 or 200, consecutive nucleotides of the nucleotide sequence according to position 1 to 591, in particular 100 to 280 of SEQ ID NO: 7;
f) polynucleotides which have a sequence identity of at least 80%, preferably at least 85, 90 or 95%, most preferably 100%, to a polynucleotide which comprises at least 10, preferably at least 15, 20, 25, 40, 60, 100, 150, 180 or 200, consecutive nucleotides of the nucleotide sequence according to position 240 to 540, in particular 300 to 540 of SEQ ID NO: 8;
g) polynucleotides which have a sequence identity of at least 80%, preferably at least 85, 90 or 95%, most preferably 100%, to a polynucleotide which comprises at least 10, preferably at least 15, 20, 25, 40, 60, 100, 150, 180 or 200, consecutive nucleotides of the nucleotide sequence according to position 12 to 418, in particular 240 to 400, of SEQ ID NO: 9;
h) polynucleotides which have a sequence identity of at least 80%, preferably at least 85, 90 or 95%, most preferably 100%, to a polynucleotide which comprises at least 10, preferably at least 15, 20, 25, 40, 60, 100, 150, 180 or 200, consecutive nucleotides of the nucleotide sequence according to position 673 to 5628 of SEQ ID NO: 16;
i) polynucleotides which have a sequence identity of at least 80%, preferably at least 85, 90 or 95%, most preferably 100%, to a polynucleotide which comprises at least 8 or 10, preferably at least 12, consecutive nucleotides of the nucleotide sequence according to position 14 to 25 of SEQ ID NO: 10;
j) polynucleotides which have a sequence identity of at least 80%, preferably at least 85, 90 or 95%, most preferably 100%, to a polynucleotide which comprises at least 8 or 10, preferably at least 12, 14, 16, 18 or 20, consecutive nucleotides of the nucleotide sequence according to position 44 to 65 of SEQ ID NO: 11;
k) polynucleotides which have a sequence identity of at least 80%, preferably at least 85, 90 or 95%, most preferably 100%, to a polynucleotide which comprises at least 8 or 10, preferably at least 12, 14, 16, 18 or 20, consecutive nucleotides of the nucleotide sequence according to position 3 to 24 of SEQ ID NO: 12;
l) polynucleotides which have a sequence identity of at least 80%, preferably at least 85, 90 or 95%, most preferably 100%, to a polynucleotide which comprises at least 8 or 10, preferably at least 12, 14, 16, 18 or 20, consecutive nucleotides of the nucleotide sequence according to position 45 to 66 of SEQ ID NO: 13,
m) polynucleotides (DNAs and RNAs) which are complementary to the sequences according to (a) to (l);
n) polynucleotides (DNAs and RNAs) which are complementary to the sequences according to (m);
o) polynucleotides which comprise the polynucleotides according to (a) to (n).

More preferably the sequences are selected from the following group (“List III”):
a) polynucleotides which have a sequence identity of at least 80%, preferably at least 85, 90 or 95%, most preferably 100%, to a polynucleotide which comprises at least 20, preferably at least 30 or 40, consecutive nucleotides of the nucleotide sequence according to position 460 to 560 of SEQ ID NO: 1,
b) polynucleotides which have a sequence identity of at least 80%, preferably at least 85, 90 or 95%, most preferably 100%, to a polynucleotide which comprises at least 20, preferably at least 30 or 40, consecutive nucleotides of the nucleotide sequence according to position 670 to 760 of SEQ ID NO: 3,
c) polynucleotides which have a sequence identity of at least 80%, preferably at least 85, 90 or 95%, most preferably 100%, to a polynucleotide which comprises at least 20, preferably at least 30 or 40, consecutive nucleotides of the nucleotide sequence according to position 80 to 220 of SEQ ID NO: 4;
d) polynucleotides which have a sequence identity of at least 80%, preferably at least 85, 90 or 95%, most preferably 100%, to a polynucleotide which comprises at least 20, preferably at least 30 or 40, consecutive nucleotides of the nucleotide sequence according to position 1400 to 1700 of SEQ ID NO: 5;
e) polynucleotides which have a sequence identity of at least 80%, preferably at least 85, 90 or 95%, most preferably 100%, to a polynucleotide which comprises at least 20, preferably at least 30 or 40, consecutive nucleotides of the nucleotide sequence according to position 100 to 280 of SEQ ID NO: 7;
f) polynucleotides which have a sequence identity of at least 80%, preferably at least 85, 90 or 95%, most preferably 100%, to a polynucleotide which comprises at least 20, preferably at least 30 or 40, consecutive nucleotides of the nucleotide sequence according to position 300 to 540 of SEQ ID NO: 8;
g) polynucleotides which have a sequence identity of at least 80%, preferably at least 85, 90 or 95%, most preferably 100%, to a polynucleotide which comprises at least 20, preferably at least 30 or 40, consecutive nucleotides of the nucleotide sequence according to position 240 to 400 of SEQ ID NO: 9;
h) polynucleotides which have a sequence identity of at least 80%, preferably at least 85, 90 or 95%, most preferably 100%, to a polynucleotide which comprises at least 20, preferably at least 30 or 40, consecutive nucleotides of the nucleotide sequence according to position 673 to 5628 of SEQ ID NO: 16;
i) polynucleotides which have a sequence identity of at least 80%, preferably at least 85, 90 or 95%, most preferably 100%, to a polynucleotide which comprises at least 8 or 10, preferably at least 12, consecutive nucleotides of the nucleotide sequence according to position 14 to 25 of SEQ ID NO: 10;
j) polynucleotides which have a sequence identity of at least 80%, preferably at least 85, 90 or 95%, most preferably 100%, to a polynucleotide which comprises at least 8 or 10, preferably at least 12, 14, 16, 18 or 20, consecutive nucleotides of the nucleotide sequence according to position 44 to 65 of SEQ ID NO: 11;
k) polynucleotides which have a sequence identity of at least 80%, preferably at least 85, 90 or 95%, most preferably 100%, to a polynucleotide which comprises at least 8 or 10, preferably at least 12, 14, 16, 18 or 20, consecutive nucleotides of the nucleotide sequence according to position 3 to 24 of SEQ ID NO: 12;
l) polynucleotides which have a sequence identity of at least 80%, preferably at least 85, 90 or 95%, most preferably 100%, to a polynucleotide which comprises at least 8 or 10, preferably at least 12, 14, 16, 18 or 20, consecutive nucleotides of the nucleotide sequence according to position 45 to 66 of SEQ ID NO: 13,
m) polynucleotides (DNAs and RNAs) which are complementary to the sequences according to (a) to (l);
n) polynucleotides (DNAs and RNAs) which are complementary to the sequences according to (m);
o) polynucleotides which comprise the polynucleotides according to (a) to (n).

In a preferred embodiment of the invention combinations of said polynucleotides/markers, preferably combinations of at least 2, 3, 4, 5, 6 or 7 of said markers are used for detecting necrotic enteritis.

Another subject of the current invention is a method of detecting infection associated, in particular necrotic enteritis associated, Clostridium perfringens, the method comprising isolating microvesicles from an avian sample, and subsequently determining the presence and/or level of at least one marker indicative for the presence of infection associated, in particular necrotic enteritis associated, Clostridium perfringens in these microvesicles, wherein the presence and/or an increased level of this at least one marker in comparison to a non-infected control is indicative for the disease, in particular necrotic enteritis. In this method the at least one marker is preferably selected from the polynucleotides of List I, (a) to (h), as well as from polynucleotides (DNAs and RNAs) complementary to those sequences. More preferably the marker is selected from the polynucleotides of List II, (a) to (h), as well as from polynucleotides (DNAs and RNAs) complementary to those sequences. Even more preferably the marker is selected from the polynucleotides of List III, (a) to (h), as well as from polynucleotides (DNAs and RNAs) complementary to those sequences.

Another subject of the current invention is a method of detecting infection associated, in particular necrotic enteritis associated, avian miRNA, the method comprising isolating microvesicles from an avian sample, and subsequently determining the presence and/or level of infection associated, in particular necrotic enteritis associated, miRNA in these microvesicles, wherein the presence and/or an increased level of this at least one marker in comparison to a non-infected control is indicative for the disease, in particular necrotic enteritis. In this method the at least one marker is preferably selected from the polynucleotides of List I, (i) to (l), as well as from polynucleotides (DNAs and RNAs) complementary to those sequences. More preferably the marker is selected from the polynucleotides of List II, (i) to (l), as well as from polynucleotides (DNAs and RNAs) complementary to those sequences. Even more preferably the marker is selected from the polynucleotides of List III, (i) to (l), as well as from polynucleotides (DNAs and RNAs) complementary to those sequences.

As it is surprising that the SAM region (SEQ ID NO: 1), the swim zinc finger region (SEQ ID NO: 3), the scRNA (SEQ ID NO: 4), the CPE0956 region (SEQ ID NO: 7), the virX region (SEQ ID NO: 9) as well as mir-16 (SEQ ID NO: 10), mir-24 (SEQ ID NO: 11), mir-155 (SEQ ID NO: 12), and mir-206 (SEQ ID NO: 13) can be used as markers for determining avian necrotic enteritis, at all, a further subject of the current invention is a method of detecting avian necrotic enteritis, in particular sub-clinical avian necrotic enteritis, wherein detection of necrotic enteritis is carried out by detecting the presence and/or amount of at least one of the following sequences (“List IV”) in an avian sample, preferably in microvesicles of an avian sample, in particular in comparison to a control sample of a non-infected avian, wherein the presence and/or an increased level of at least one of those sequences in comparison to a non-infected control is indicative for necrotic enteritis:
a) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence as depicted in SEQ ID NO: 1,
b) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence as depicted in SEQ ID NO: 3,
c) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence as depicted in SEQ ID NO: 4;
d) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence as depicted in SEQ ID NO: 7;
e) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence as depicted in SEQ ID NO: 9;
f) polynucleotides which have a sequence identity of at least 80%, preferably at least 85, 90 or 95%, most preferably 100%, to a polynucleotide which comprises at least 8 or 10, preferably at least 12, 14, 16, 18, 20, 25, 40, 60 or 80, consecutive nucleotides of the nucleotide sequence as depicted in SEQ ID NO: 10;
g) polynucleotides which have a sequence identity of at least 80%, preferably at least 85, 90 or 95%, most preferably 100%, to a polynucleotide which comprises at least 8 or 10, preferably at least 12, 14, 16, 18, 20, 25, 40 or 60, consecutive nucleotides of the nucleotide sequence as depicted in SEQ ID NO: 11;
h) polynucleotides which have a sequence identity of at least 80%, preferably at least 85, 90 or 95%, most preferably 100%, to a polynucleotide which comprises at least 8 or 10, preferably at least 12, 14, 16, 18, 20, 25, 40 or 60, consecutive nucleotides of the nucleotide sequence as depicted in SEQ ID NO: 12;
i) polynucleotides which have a sequence identity of at least 80%, preferably at least 85, 90 or 95%, most preferably 100%, to a polynucleotide which comprises at least 8 or 10, preferably at least 12, 14, 16, 18, 20, 25, 40 or 60, consecutive nucleotides of the nucleotide sequence as depicted in SEQ ID NO: 13;
j) polynucleotides (DNAs and RNAs) which are complementary to the sequences according to (a) to (i);
k) polynucleotides (DNAs and RNAs) which are complementary to the sequences according to (j);
l) polynucleotides which comprise the polynucleotides according to (a) to (k).

In this method the sequences are preferably selected from the following group (“List V”):
a) polynucleotides which have a sequence identity of at least 80%, preferably at least 85, 90 or 95%, most preferably 100%, to a polynucleotide which comprises at least 20, preferably at least 25, 40, 60, 100, 150, 180 or 200 consecutive nucleotides of the nucleotide sequence according to position 1 to 630, in particular 420 to 630, preferably 460 to 560, of SEQ ID NO: 1,
b) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence according to position 600 to 1000, in particular 670 to 760, of SEQ ID NO: 3,
c) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence according to position 60 to 240, in particular 80 to 220, of SEQ ID NO: 4;
d) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence according to position 1 to 591, in particular 100 to 280 of SEQ ID NO: 7;
e) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence according to position 12 to 418, in particular 240 to 400 of SEQ ID NO: 9;
f) polynucleotides which have a sequence identity of at least 80%, preferably at least 85, 90 or 95%, most preferably 100%, to a polynucleotide which comprises at least 8 or 10, preferably at least 12, consecutive nucleotides of the nucleotide sequence according to position 14 to 25 of SEQ ID NO: 10;
g) polynucleotides which have a sequence identity of at least 80%, preferably at least 85, 90 or 95%, most preferably 100%, to a polynucleotide which comprises at least 8 or 10, preferably at least 12, 14, 16, 18 or 20, consecutive nucleotides of the nucleotide sequence according to position 44 to 65 of SEQ ID NO: 11;
h) polynucleotides which have a sequence identity of at least 80%, preferably at least 85, 90 or 95%, most preferably 100%, to a polynucleotide which comprises at least 8 or 10, preferably at least 12, 14, 16, 18 or 20, consecutive nucleotides of the nucleotide sequence according to position 3 to 24 of SEQ ID NO: 12;
i) polynucleotides which have a sequence identity of at least 80%, preferably at least 85, 90 or 95%, most preferably 100%, to a polynucleotide which comprises at least 8 or 10, preferably at least 12, 14, 16, 18 or 20, consecutive nucleotides of the nucleotide sequence according to position 45 to 66 of SEQ ID NO: 13,
j) polynucleotides (DNAs and RNAs) which are complementary to the sequences according to (a) to (i);
k) polynucleotides (DNAs and RNAs) which are complementary to the sequences according to (j);
l) polynucleotides which comprise the polynucleotides according to (a) to (k).

More preferably the sequences are selected from the following group (“List VI”):
a) polynucleotides which have a sequence identity of at least 90%, preferably at least 95, 98 or 99%, most preferably 100%, to a polynucleotide which comprises at least 20, preferably at least 30 or 40, consecutive nucleotides of the nucleotide sequence according to position 460 to 560 of SEQ ID NO: 1,
b) polynucleotides which have a sequence identity of at least 90%, preferably at least 95, 98 or 99%, most preferably 100%, to a polynucleotide which comprises at least 20, preferably at least 30 or 40, consecutive nucleotides of the nucleotide sequence according to position 670 to 760 of SEQ ID NO: 3,
c) polynucleotides which have a sequence identity of at least 90%, preferably at least 95, 98 or 99%, most preferably 100%, to a polynucleotide which comprises at least 20, preferably at least 30 or 40, consecutive nucleotides of the nucleotide sequence according to position 80 to 220 of SEQ ID NO: 4;
d) polynucleotides which have a sequence identity of at least 90%, preferably at least 95, 98 or 99%, most preferably 100%, to a polynucleotide which comprises at least 20, preferably at least 30 or 40, consecutive nucleotides of the nucleotide sequence according to position 100 to 280 of SEQ ID NO: 7;
e) polynucleotides which have a sequence identity of at least 90%, preferably at least 95, 98 or 99%, most preferably 100%, to a polynucleotide which comprises at least 20, preferably at least 30 or 40, consecutive nucleotides of the nucleotide sequence according to position 240 to 400 of SEQ ID NO: 9;
f) polynucleotides which have a sequence identity of at least 90%, preferably at least 95, 98 or 99%, most preferably 100%, to a polynucleotide which comprises at least 8 or 10, preferably at least 12, consecutive nucleotides of the nucleotide sequence according to position 14 to 25 of SEQ ID NO: 10;
g) polynucleotides which have a sequence identity of at least 90%, preferably at least 95, 98 or 99%, most preferably 100%, to a polynucleotide which comprises at least 8 or 10, preferably at least 12, 14, 16, 18 or 20, consecutive nucleotides of the nucleotide sequence according to position 44 to 65 of SEQ ID NO: 11;
h) polynucleotides which have a sequence identity of at least 90%, preferably at least 95, 98 or 99%, most preferably 100%, to a polynucleotide which comprises at least 8 or 10, preferably at least 12, 14, 16, 18 or 20, consecutive nucleotides of the nucleotide sequence according to position 3 to 24 of SEQ ID NO: 12;
i) polynucleotides which have a sequence identity of at least 90%, preferably at least 95, 98 or 99%, most preferably 100%, to a polynucleotide which comprises at least 8 or 10, preferably at least 12, 14, 16, 18 or 20, consecutive nucleotides of the nucleotide sequence according to position 45 to 66 of SEQ ID NO: 13,
j) polynucleotides (DNAs and RNAs) which are complementary to the sequences according to (a) to (i);
k) polynucleotides (DNAs and RNAs) which are complementary to the sequences according to (j);
l) polynucleotides which comprise the polynucleotides according to (a) to (k).

In a preferred embodiment of the invention combinations of said polynucleotides, preferably combinations of at least 2, 3, 4, 5, 6 or 7 of said polynucleotides are used for detecting necrotic enteritis.

The avian sample according to the invention can be a tissue sample, preferably an intestinal sample, in particular a duodenal or a jejunal sample.

Preferably the avian sample is selected from avian bodily fluids and avian excrements and solutions and suspensions thereof.

The avian bodily fluid according to the invention is preferably blood, blood serum or blood plasma.

The avian excrement according to the invention is preferably selected from fecal and cecal excrements.

Suitable sample volumes are, for example, 0.1 to 20 ml, in particular 0.2 to 10 ml, preferably 0.5 to 5 ml. Suitable sample masses are, for example 0.1 to 20 g, in particular 0.2 to 10 g, preferably 0.5 to 5 g.

The avian subject according to the invention is preferably poultry.

Preferred poultry according to the invention are chickens, turkeys, ducks and geese. The poultry can be optimized for producing young stock. This type of poultry is also referred to as parent animals. Preferred parent animals are, accordingly, parent broilers, parent ducks, parent turkeys and parent geese.

The poultry according to the invention can also be selected from fancy poultry and wild fowl.

Preferred fancy poultry or wild fowl are peacocks, pheasants, partridges, guinea fowl, quails, capercaillies, grouse, pigeons and swans.

Further preferred poultry according to the invention are ostriches and parrots.

Most preferred poultry according to the invention are chickens.

As used herein, the term “polynucleotides” or “nucleic acids” refer to DNA and RNA. The polynucleotides and nucleic acids can be single stranded or double stranded.

The isolation of microvesicles from avian excrements and its subsequent use to determine an avian infection, in particular avian bacterial infection, more particular necrotic enteritis, has not been disclosed before.

Thus, a further subject of the current invention is a method of detecting an avian infection, in particular a sub-clinical avian infection, preferably an avian bacterial infection, in particular a sub-clinical avian bacterial infection, more preferably necrotic enteritis, in particular sub-clinical necrotic enteritis, the method comprising determining the presence and/or amount of at least one marker indicative for the disease in microvesicles isolated from avian excrements, in particular from fecal or cecal excrements, wherein the presence and/or an increased level of this at least one marker in comparison to a non-infected control is indicative for the disease. In this method the at least one marker is preferably selected from any of the polynucleotides as disclosed in List I, preferably as disclosed in List II, more preferably as disclosed in List Ill.

It turned out that the occurrence of markers indicative for necrotic enteritis is sample-specific, i.e. the detectable amount of the different markers is dependent on the sample type.

So it was found that in fecal excrements the best marker is the SAM region, followed by scRNA, the netB region, virX region, colA region, swim zinc finger region and the CPE0956 region.

To the contrary, in cecal excrements the best marker is also the SAM region, but is followed by the swim zinc finger region, scRNA, mir206, the virX region, the netB region, the CPE0956 region and the colA region.

Further in blood samples the best marker is mir16, followed by mir155 and mir24.

Thus a preferred embodiment of the current application is a method of detecting avian necrotic enteritis, in particular sub-clinical avian necrotic enteritis, the method comprising determining the presence and/or the level of at least one marker indicative for necrotic enteritis in avian fecal excrements, preferably in microvesicles isolated from avian fecal excrements, wherein the presence and/or an increased level of this at least one marker in comparison to a non-infected control is indicative for necrotic enteritis and wherein the marker is preferably selected from
a) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence as depicted in SEQ ID NO: 1,
b) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence as depicted in SEQ ID NO: 3,
c) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence as depicted in SEQ ID NO: 4;
d) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence as depicted in SEQ ID NO: 5;
e) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence as depicted in SEQ ID NO: 7;
f) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence as depicted in SEQ ID NO: 8;
g) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence as depicted in SEQ ID NO: 9;
h) polynucleotides (DNAs and RNAs) which are complementary to the sequences according to (a) to (g);
i) polynucleotides (DNAs and RNAs) which are complementary to the sequences according to (h);
j) polynucleotides which comprise the polynucleotides according to (a) to (i).

In this method the sequences are preferably selected from the following group:
a) polynucleotides which have a sequence identity of at least 80%, preferably at least 85, 90 or 95%, most preferably 100%, to a polynucleotide which comprises at least 20, preferably at least 25, 40, 60 or 100, consecutive nucleotides of the nucleotide sequence according to position 1 to 630, in particular 420 to 630, preferably 460 to 560, of SEQ ID NO: 1,
b) polynucleotides which have a sequence identity of at least 80%, preferably at least 85, 90 or 95%, most preferably 100%, to a polynucleotide which comprises at least 20, preferably at least 25, 40, 60 or 90, consecutive nucleotides of the nucleotide sequence according to position 600 to 1000, in particular 670 to 760, of SEQ ID NO: 3,
c) polynucleotides which have a sequence identity of at least 80%, preferably at least 85, 90 or 95%, most preferably 100%, to a polynucleotide which comprises at least 20, preferably at least 25, 40, 60, 100 or 140, consecutive nucleotides of the nucleotide sequence according to position 60 to 240, in particular 80 to 220, of SEQ ID NO: 4;
d) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides according to position 1300 to 1800, in particular 1400 to 1700, of SEQ ID NO: 5;
e) polynucleotides which have a sequence identity of at least 80%, preferably at least 85, 90 or 95%, most preferably 100%, to a polynucleotide which comprises at least 20, preferably at least 25, 40, 60, 100, 150 or 180, consecutive nucleotides of the nucleotide sequence according to position 1 to 591, in particular 100 to 280 of SEQ ID NO: 7;
f) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence according to position 240 to 540, in particular 300 to 540 of SEQ ID NO: 8;
g) polynucleotides which have a sequence identity of at least 80%, preferably at least 85, 90 or 95%, most preferably 100%, to a polynucleotide which comprises at least 20, preferably at least 25, 40, 60, 100, 150 or 160, consecutive nucleotides of the nucleotide sequence according to position 12 to 418, in particular 240 to 400 of SEQ ID NO: 9;
h) polynucleotides (DNAs and RNAs) which are complementary to the sequences according to (a) to (g);
i) polynucleotides (DNAs and RNAs) which are complementary to the sequences according to (h);
j) polynucleotides which comprise the polynucleotides according to (a) to (i).

Thus another preferred embodiment of the current application is a method of detecting avian necrotic enteritis, in particular sub-clinical avian necrotic enteritis, the method comprising determining the presence and/or level of at least one marker indicative for necrotic enteritis in avian cecal excrements, in particular in microvesicles isolated from avian cecal excrements, wherein the presence and/or an increased level of this at least one marker in comparison to a non-infected control is indicative for necrotic enteritis and wherein the marker is preferably selected from
a) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence as depicted in SEQ ID NO: 1,
b) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence as depicted in SEQ ID NO: 3,
c) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence as depicted in SEQ ID NO: 4;
d) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence as depicted in SEQ ID NO: 5;
e) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence as depicted in SEQ ID NO: 7;
f) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence as depicted in SEQ ID NO: 8;
g) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence as depicted in SEQ ID NO: 9;
h) polynucleotides which have a sequence identity of at least 80%, preferably at least 85, 90 or 95%, most preferably 100%, to a polynucleotide which comprises at least 8 or 10, preferably at least 12, 14, 16, 18, 20, 25, 40 or 60, consecutive nucleotides of the nucleotide sequence as depicted in SEQ ID NO: 13;
i) polynucleotides (DNAs and RNAs) which are complementary to the sequences according to (a) to (h);
j) polynucleotides (DNAs and RNAs) which are complementary to the sequences according to (i);
k) polynucleotides which comprise the polynucleotides according to (a) to (j).

In this method the sequences are preferably selected from the following group:
a) polynucleotides which have a sequence identity of at least 80%, preferably at least 85, 90 or 95%, most preferably 100%, to a polynucleotide which comprises at least 20, preferably at least 25, 40, 60 or 100, consecutive nucleotides of the nucleotide sequence according to position 1 to 630, in particular 420 to 630, preferably 460 to 560, of SEQ ID NO: 1,
b) polynucleotides which have a sequence identity of at least 80%, preferably at least 85, 90 or 95%, most preferably 100%, to a polynucleotide which comprises at least 20, preferably at least 25, 40, 60 or 90, consecutive nucleotides of the nucleotide sequence according to position 600 to 1000, in particular 670 to 760, of SEQ ID NO: 3,
c) polynucleotides which have a sequence identity of at least 80%, preferably at least 85, 90 or 95%, most preferably 100%, to a polynucleotide which comprises at least 20, preferably at least 25, 40, 60, 100 or 140, consecutive nucleotides of the nucleotide sequence according to position 60 to 240, in particular 80 to 220, of SEQ ID NO: 4;
d) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides according to position 1300 to 1800, in particular 1400 to 1700, of SEQ ID NO: 5;
e) polynucleotides which have a sequence identity of at least 80%, preferably at least 85, 90 or 95%, most preferably 100%, to a polynucleotide which comprises at least 20, preferably at least 25, 40, 60, 100, 150 or 180, consecutive nucleotides of the nucleotide sequence according to position 1 to 591, in particular 100 to 280 of SEQ ID NO: 7;
f) polynucleotides which have a sequence identity of at least 80%, preferably at least 85, 90 or 95%, most preferably 100%, to a polynucleotide which comprises at least 20, preferably at least 25, 40, 60, 100, 150, 180 or 200, consecutive nucleotides of the nucleotide sequence according to position 240 to 540, in particular 300 to 540 of SEQ ID NO: 8;
g) polynucleotides which have a sequence identity of at least 80%, preferably at least 85, 90 or 95%, most preferably 100%, to a polynucleotide which comprises at least 20, preferably at least 25, 40, 60, 100, 150 or 160, consecutive nucleotides of the nucleotide sequence according to position 12 to 418, in particular 240 to 400 of SEQ ID NO: 9;
h) polynucleotides which have a sequence identity of at least 80%, preferably at least 85, 90 or 95%, most preferably 100%, to a polynucleotide which comprises at least 8 or 10, preferably at least 12, 14, 16, 18 or 20, consecutive nucleotides of the nucleotide sequence according to position 45 to 66 of SEQ ID NO: 13,
i) polynucleotides (DNAs and RNAs) which are complementary to the sequences according to (a) to (h);
j) polynucleotides (DNAs and RNAs) which are complementary to the sequences according to (i);
k) polynucleotides which comprise the polynucleotides according to (a) to (j).

Thus another preferred embodiment of the current application is a method of detecting avian necrotic enteritis, in particular sub-clinical necrotic enteritis, the method comprising determining the presence and/or level of at least one marker indicative for necrotic enteritis in avian blood, in particular in microvesicles isolated from avian blood, wherein the presence and/or an increased level of this at least one marker in comparison to a non-infected control is indicative for necrotic enteritis and wherein the at least one marker is preferably selected from
a) polynucleotides which have a sequence identity of at least 80%, preferably at least 85, 90 or 95%, most preferably 100%, to a polynucleotide which comprises at least 8 or 10, preferably at least 12, 14, 16, 18, 20, 25, 40, 60 or 80, consecutive nucleotides of the nucleotide sequence as depicted in SEQ ID NO: 10;
b) polynucleotides which have a sequence identity of at least 80%, preferably at least 85, 90 or 95%, most preferably 100%, to a polynucleotide which comprises at least 8 or 10, preferably at least 12, 14, 16, 18, 20, 25, 40 or 60, consecutive nucleotides of the nucleotide sequence as depicted in SEQ ID NO: 11;
c) polynucleotides which have a sequence identity of at least 80%, preferably at least 85, 90 or 95%, most preferably 100%, to a polynucleotide which comprises at least 8 or 10, preferably at least 12, 14, 16, 18, 20, 25, 40 or 60, consecutive nucleotides of the nucleotide sequence as depicted in SEQ ID NO: 12;
d) polynucleotides (DNAs and RNAs) which are complementary to the sequences according to (a) to (c);
e) polynucleotides (DNAs and RNAs) which are complementary to the sequences according to (d);
f) polynucleotides which comprise the polynucleotides according to (a) to (e).

In this method the sequences are preferably selected from the following group:
a) polynucleotides which have a sequence identity of at least 80%, preferably at least 85, 90 or 95%, most preferably 100%, to a polynucleotide which comprises at least 8 or 10, preferably at least 12, consecutive nucleotides of the nucleotide sequence according to position 14 to 25 of SEQ ID NO: 10;
b) polynucleotides which have a sequence identity of at least 80%, preferably at least 85, 90 or 95%, most preferably 100%, to a polynucleotide which comprises at least 8 or 10, preferably at least 12, 14, 16, 18 or 20, consecutive nucleotides of the nucleotide sequence according to position 44 to 65 of SEQ ID NO: 11;
c) polynucleotides which have a sequence identity of at least 80%, preferably at least 85, 90 or 95%, most preferably 100%, to a polynucleotide which comprises at least 8 or 10, preferably at least 12, 14, 16, 18 or 20, consecutive nucleotides of the nucleotide sequence according to position 3 to 24 of SEQ ID NO: 12;
d) polynucleotides (DNAs and RNAs) which are complementary to the sequences according to (a) to (c);
e) polynucleotides (DNAs and RNAs) which are complementary to the sequences according to (d);
f) polynucleotides which comprise the polynucleotides according to (a) to (e).

A further subject of the current invention is a method of treatment of necrotic enteritis comprising performing the method of detecting necrotic enteritis as described before and subsequently administering a therapeutic agent for the treatment of necrotic enteritis.

A further subject of the current invention is also a nucleotide array for detecting avian necrotic enteritis, wherein the nucleotide array comprises at least one marker, preferably at least 2, 3, 4 or 5 markers, selected from the polynucleotides as depicted in List IV, preferably as depicted in List V, more preferably as depicted in List VI.

A further subject of the current invention is also a kit comprising a set of oligonucleotides for amplifying at least one polynucleotide associated with necrotic enteritis, wherein the at least one polynucleotide is selected from the polynucleotides as depicted in List IV, preferably as depicted in List V, more preferably as depicted in List VI.

NetB and TpeL have already been discussed to be major toxins involved in the pathogenesis of necrotic enteritis (Keyburn et al, 2010; WO 2008/148166; Shojadoost et al., 2012)

ColA stands for Collagenese A of Clostridium perfringens; this gene might be associated with the pathogenesis of NE because of its similarity to the so called kappa-toxin, one of the supposed major toxins of this bacterium (Obana et al., 2013).

Nevertheless, the occurrence as well as the deregulation of these genes in microvesicles could not have been expected.

The virX gene is known to regulate the plx, colA, and pfoA genes in C. perfringens (Ohtani et al., 2002), but its involvement in the pathogenesis of NE has not been reported before.

The small cytoplasmic RNA (scRNA), a member of an evolutionarily conserved signal-recognition-particle-like RNA family involved in the sporulation mechanism of Clostridium perfringens (Nakamura et al., 1995) has also not been reported previously to be in direct association with NE in chickens.

Furthermore, the involvement of the SAM domain, the swim zinc finger domain and the CPE0956 region in the pathogenesis of necrotic enteritis has not been disclosed before and could also not have been expected as these sequences are sequences with previously unknown functions.

A further subject of the current inventions are therefore polynucleotides, preferably encoding a polypeptide with toxin activity and/or a virulence factor involved in necrotic enteritis, selected from the group consisting of
a) polynucleotides which have a sequence identity of at least 80%, preferably at least 85, 90 or 95%, most preferably 100%, to a polynucleotide which comprises at least 100, preferably at least 150, 180 or 200, consecutive nucleotides of the nucleotide sequence as depicted in SEQ ID NO: 1;
b) polynucleotides which have a sequence identity of at least 90%, preferably at least 95, 98 or 99%, most preferably 100%, to a polynucleotide which comprises at least 20, preferably at least 25, 40, 60, 100, 150, 180 or 200, consecutive nucleotides of the nucleotide sequence as depicted in SEQ ID NO: 1;
c) polynucleotides which have a sequence identity of at least 80%, preferably at least 85, 90 or 95%, most preferably 100%, to a polynucleotide which comprises at least 100, preferably at least 150, 180 or 200, consecutive nucleotides of the nucleotide sequence as depicted in SEQ ID NO: 3;
d) polynucleotides which have a sequence identity of at least 90%, preferably at least 95, 98 or 99%, most preferably 100%, to a polynucleotide which comprises at least 20, preferably at least 25, 40, 60, 100, 150, 180 or 200, consecutive nucleotides of the nucleotide sequence as depicted in SEQ ID NO: 3;
e) polynucleotides which have a sequence identity of at least 80%, preferably at least 85, 90 or 95%, most preferably 100%, to a polynucleotide which comprises at least 100, preferably at least 150, 180 or 200, consecutive nucleotides of the nucleotide sequence as depicted in SEQ ID NO: 7;
f) polynucleotides which have a sequence identity of at least 90%, preferably at least 95, 98 or 99%, most preferably 100%, to a polynucleotide which comprises at least 20, preferably at least 25, 40, 60, 100, 150, 180 or 200, consecutive nucleotides of the nucleotide sequence as depicted in SEQ ID NO: 7;
g) polynucleotides (DNAs and RNAs) which are complementary to the sequences according to (a) to (f);
h) polynucleotides which comprise the polynucleotides according to (a) to (g).

Particularly preferred are polynucleotides selected from the group consisting of
a) polynucleotides which have a sequence identity of at least 80%, preferably at least 85, 90 or 95%, most preferably 100%, to a polynucleotide which comprises at least 100, preferably at least 150, 180 or 200, consecutive nucleotides of the nucleotide sequence according to position 1 to 630, in particular 420 to 630, preferably 460 to 560, of SEQ ID NO: 1;
b) polynucleotides which have a sequence identity of at least 90%, preferably at least 95, 98 or 99%, most preferably 100%, to a polynucleotide which comprises at least 10, preferably at least 15, 20, 25, 40, 60, 100, 150, 180 or 200, consecutive nucleotides of the nucleotide sequence according to position 1 to 630, in particular 420 to 630, preferably 460 to 560, of SEQ ID NO: 1;
c) polynucleotides which have a sequence identity of at least 80%, preferably at least 85, 90 or 95%, most preferably 100%, to a polynucleotide which comprises at least 100, preferably at least 150, 180 or 200, consecutive nucleotides of the nucleotide sequence according to position 600 to 1000, in particular 670 to 760, of SEQ ID NO: 3;
d) polynucleotides which have a sequence identity of at least 90%, preferably at least 95, 98 or 99%, most preferably 100%, to a polynucleotide which comprises at least 20, preferably at least 25, 40, 60, 100, 150, 180 or 200, consecutive nucleotides of the nucleotide sequence according to position 600 to 1000, in particular 670 to 760, of SEQ ID NO: 3;
e) polynucleotides which have a sequence identity of at least 80%, preferably at least 85, 90 or 95%, most preferably 100%, to a polynucleotide which comprises at least 100, preferably at least 150, 180 or 200, consecutive nucleotides of the nucleotide sequence according to position 1 to 591, in particular 100 to 280 of SEQ ID NO: 7;
f) polynucleotides which have a sequence identity of at least 90%, preferably at least 95, 98 or 99%, most preferably 100%, to a polynucleotide which comprises at least 20, preferably at least 25, 40, 60, 100, 150, 180 or 200, consecutive nucleotides of the nucleotide sequence according to position 1 to 591, in particular 100 to 280 of SEQ ID NO: 7;
g) polynucleotides (DNAs and RNAs) which are complementary to the sequences according to (a) to (f);
h) polynucleotides which comprise the polynucleotides according to (a) to (g).

A further subject of the current invention are therefore also polypeptides, which are preferably toxins and/or virulence factors involved in necrotic enteritis, selected from the group consisting of
a) polypeptides which have a sequence identity of at least 80%, preferably at least 85, 90 or 95%, most preferably 100%, to a polypeptide which comprises at least 10, preferably at least 15, 20, 25, 40, 60, 100, 150, 180 or 200, in particular all, consecutive amino acids of the amino acid sequence as depicted in SEQ ID NO: 2;
b) polypeptides which have a sequence identity of at least 80%, preferably at least 85, 90 or 95%, most preferably 100%, to a polypeptide which comprises at least 10, preferably at least 15, 20, 25, 40, 60 or 70, in particular all, consecutive amino acids of the amino acid sequence as depicted in SEQ ID NO: 14;
c) polypeptides which have a sequence identity of at least 80%, preferably at least 85, 90 or 95%, most preferably 100%, to a polypeptide which comprises at least 10, preferably at least 15, 20, 25, 40, 60, 100, 150, 180 or 190, in particular all, consecutive amino acids of the amino acid sequence as depicted in SEQ ID NO: 15;
d) polypeptides which comprise the polypeptides according to (a) to (c).

Particularly preferred are polypeptides selected from the group consisting of
a) polypeptides which have a sequence identity of at least 80%, preferably at least 85, 90 or 95%, most preferably 100%, to a polypeptide which comprises at least 100, preferably at least 150, 180 or 200, in particular all, consecutive amino acids of the amino acid sequence as depicted in SEQ ID NO: 2;
b) polypeptides which have a sequence identity of at least 80%, preferably at least 85, 90 or 95%, most preferably 100%, to a polypeptide which comprises at least 40, preferably at least 60 or 70, in particular all, consecutive amino acids of the amino acid sequence as depicted in SEQ ID NO: 14;
c) polypeptides which have a sequence identity of at least 80%, preferably at least 85, 90 or 95%, most preferably 100%, to a polypeptide which comprises at least 100, preferably at least 150, 180 or 190, in particular all, consecutive amino acids of the amino acid sequence as depicted in SEQ ID NO: 15;
d) polypeptides which comprise the polypeptides according to (a) to (c).

A further subject of the current invention is therefore also a method of raising an immune response in an animal; the method comprising administering to the animal a polypeptide as mentioned before, in particular selected from the group consisting of
a) polypeptides which have a sequence identity of at least 80%, preferably at least 85, 90 or 95%, most preferably 100%, to a polypeptide which comprises at least 10, preferably at least 15, 20, 25, 40, 60, 100, 150, 180 or 200, in particular all, consecutive amino acids of the amino acid sequence as depicted in SEQ ID NO: 2;
b) polypeptides which have a sequence identity of at least 80%, preferably at least 85, 90 or 95%, most preferably 100%, to a polypeptide which comprises at least 10, preferably at least 15, 20, 25, 40, 60 or 70, in particular all, consecutive amino acids of the amino acid sequence as depicted in SEQ ID NO: 14;
c) polypeptides which have a sequence identity of at least 80%, preferably at least 85, 90 or 95%, most preferably 100%, to a polypeptide which comprises at least 10, preferably at least 15, 20, 25, 40, 60, 100, 150, 180 or 190, in particular all, consecutive amino acids of the amino acid sequence as depicted in SEQ ID NO: 15;
d) polypeptides which comprise the polypeptides according to (a) to (c).

The polypeptides according to the invention preferably exhibit toxin activity and are preferably obtainable from Clostridium perfringens. More preferably the polypeptides according to the invention are NE associated virulence factors.

A further subject of the current invention are vectors, in particular cloning and expression vectors as well as viral and plasmid vectors, comprising a polynucleotide according to the invention.

A further subject of the current invention are also cells, in particular bacterial cells, preferably E. coli cells, comprising a polynucleotide, a polypeptide and/or a vector according to the invention.

A further subject of the current invention is a polyclonal or monoclonal antibody or a fragment thereof, which binds specifically to a polypeptide with toxin activity according to the invention, in particular to NE associated virulence factors according to the invention.

A further subject of the current invention is a composition, comprising a polynucleotide, a polypeptide, a vector, a cell or an antibody according to the invention.

A further subject of the current invention is a diagnostic kit comprising a polynucleotide or an antibody according to the invention. The kit may further comprise a capture surface apparatus suitable to separate microvesicles from a biological sample from unwanted particles, debris, and small molecules, as explained in more detail below. The kit might also include instructions for its use, in particular for using the components in the optional isolation and lysis of microvesicles and the subsequent qualitative or quantitative determination of the presence of at least one of the markers according to the invention.

A further subject of the current invention is an immunogenic composition comprising a polypeptide according to the invention and an adjuvant and/or a pharmaceutically acceptable carrier.

A further subject of the current invention is also a vaccine, comprising a polypeptide according to the invention and preferably an adjuvant and/or a pharmaceutically acceptable carrier.

Microvesicles according to the invention comprise membrane vesicles from bacteria, in particular from Clostridium perfringens, as well as from the host, in particular chicken.

The eukaryotic version of membrane vesicles is also called exosomes. Exosomes have typically a diameter of 30-200 nm. They are shed into all biofluids and carry many intact proteins and oligonucleotides, as they are protected from degredation by RNAses (Koga et al., 2011)

Exosomes have been studied in particular in cancer and immune cells.

Microvesicles from bacteria are also called extracellular membrane vesicles (MVs) or outer membrane vesicles (OMVs). They are spherical bilayer structures with average diameter of typically 20-500 nm. Bacterial microvesicles have first been described for Gram-negative bacteria, but later also for some Gram-positive bacteria. In a recent study, it was reported that microvesicles are produced and released also by C. perfringens type A strains, triggering innate and adaptive immune responses. It was further found out in the course of this study that important virulence factors like alpha-toxin and NetB were not present in the membrane vesicles, whereas beta2-toxin was found in the membrane vesicles (Jiang et al., (2014).

MicroRNAs (miRNAs) are small, non-coding RNAs that are usually 18-23 nucleotides in size and have been suggested to play a critical role in the regulation of gene expression. In particular the genes associated with immune responses have been reported to be highly targeted by miRNAs. In chickens miRNA expression has been studied in embryo developmental processes, germ cell development, immune organs and diseases.

In recent studies the miRNA profile of two different highly inbred White Leghorn chicken lines infected by C. perfringens was investigated (Dinh et al., 2014; Hong et al., 2014). RNA analysis was carried out with tissue from spleen and intestinal mucosa. The results suggest that some miRNAs are differentially altered in response to necrotic enteritis and that they modulate the expression of their target genes. Microvesicles were not investigated in these studies.

The miRNAs as found to be correlative to necrotic enteritis according to the current invention were investigated before by Wang et al. in connection with avian influenza virus infected broilers (Wang et al., 2012). Wang et al. found that the miRNAs miR-155, miR-16-1 and miR-24 are expressed stronger in the lungs of infected broilers than in those of non-infected ones, but they found further that the miR-206 is, to the contrary, stronger expressed in non-infected ones than in infected ones. Thus the use of miR-206 as marker for a bacterial infection is disclosed in the current application for the first time. Further in contrast to Wang et al. according to the current invention all miRNAs were detected in microvesicles, not in tissue.

Thus a further subject of the current invention is a method of detecting an avian bacterial infection, in particular avian necrotic enteritis, the method comprising isolating microvesicles from an avian sample and subsequently determining the level of at least one avian marker indicative for the infection, wherein an increased level of this at least one avian marker in comparison to a non-infected control is indicative for the infection and wherein the at least one avian marker is an avian miRNA and is preferably selected from the group consisting of
a) polynucleotides which have a sequence identity of at least 80%, preferably at least 85, 90 or 95%, most preferably 100%, to a polynucleotide which comprises at least 8 or 10, preferably at least 12, 14, 16, 18, 20, 25, 40, 60 or 80, consecutive nucleotides of the nucleotide sequence as depicted in SEQ ID NO: 10;
b) polynucleotides which have a sequence identity of at least 80%, preferably at least 85, 90 or 95%, most preferably 100%, to a polynucleotide which comprises at least 8 or 10, preferably at least 12, 14, 16, 18, 20, 25, 40 or 60, consecutive nucleotides of the nucleotide sequence as depicted in SEQ ID NO: 11;
c) polynucleotides which have a sequence identity of at least 80%, preferably at least 85, 90 or 95%, most preferably 100%, to a polynucleotide which comprises at least 8 or 10, preferably at least 12, 14, 16, 18, 20, 25, 40 or 60, consecutive nucleotides of the nucleotide sequence as depicted in SEQ ID NO: 12;
d) polynucleotides which have a sequence identity of at least 80%, preferably at least 85, 90 or 95%, most preferably 100%, to a polynucleotide which comprises at least 8 or 10, preferably at least 12, 14, 16, 18, 20, 25, 40 or 60, consecutive nucleotides of the nucleotide sequence as depicted in SEQ ID NO: 13;
e) polynucleotides (DNAs and RNAs) which are complementary to the sequences according to (a) to (d);
f) polynucleotides (DNAs and RNAs) which are complementary to the sequences according to (e);
g) polynucleotides which comprise the polynucleotides according to (a) to (f).

In this method the sequences are preferably selected from the following group:
a) polynucleotides which have a sequence identity of at least 80%, preferably at least 85, 90 or 95%, most preferably 100%, to a polynucleotide which comprises at least 8 or 10, preferably at least 12, consecutive nucleotides of the nucleotide sequence according to position 14 to 25 of SEQ ID NO: 10;
b) polynucleotides which have a sequence identity of at least 80%, preferably at least 85, 90 or 95%, most preferably 100%, to a polynucleotide which comprises at least 8 or 10, preferably at least 12, 14, 16, 18 or 20, consecutive nucleotides of the nucleotide sequence according to position 44 to 65 of SEQ ID NO: 11;
c) polynucleotides which have a sequence identity of at least 80%, preferably at least 85, 90 or 95%, most preferably 100%, to a polynucleotide which comprises at least 8 or 10, preferably at least 12, 14, 16, 18 or 20, consecutive nucleotides of the nucleotide sequence according to position 3 to 24 of SEQ ID NO: 12;
d) polynucleotides which have a sequence identity of at least 80%, preferably at least 85, 90 or 95%, most preferably 100%, to a polynucleotide which comprises at least 8 or 10, preferably at least 12, 14, 16, 18 or 20, consecutive nucleotides of the nucleotide sequence according to position 45 to 66 of SEQ ID NO: 13,
e) polynucleotides (DNAs and RNAs) which are complementary to the sequences according to (a) to (d);
f) polynucleotides (DNAs and RNAs) which are complementary to the sequences according to (e);
g) polynucleotides which comprise the polynucleotides according to (a) to (f).

As mir-206 could be for the first time be identified as marker for avian infections, at all, a further subject of the current invention is a method for detecting an avian infection, preferably an avian bacterial infection, more preferably avian necrotic enteritis, wherein an increased level of at least one avian marker in comparison to a non-infected control is indicative for the infection and wherein the at least one avian marker is selected from the group consisting of
a) polynucleotides which have a sequence identity of at least 80%, preferably at least 85, 90 or 95%, most preferably 100%, to a polynucleotide which comprises at least 8 or 10, preferably at least 12, 14, 16, 18, 20, 25, 40 or 60, consecutive nucleotides of the nucleotide sequence as depicted in SEQ ID NO: 13;
b) polynucleotides (DNAs and RNAs) which are complementary to the sequences according to (a);
c) polynucleotides which comprise the polynucleotides according to (a) or (b).

In this method the sequences are preferably selected from the following group:
a) polynucleotides which have a sequence identity of at least 80%, preferably at least 85, 90 or 95%, most preferably 100%, to a polynucleotide which comprises at least 8 or 10, preferably at least 12, 14, 16, 18 or 20, consecutive nucleotides of the nucleotide sequence according to position 45 to 66 of SEQ ID NO: 13,
b) polynucleotides (DNAs and RNAs) which are complementary to the sequences according to (a);
c) polynucleotides which comprise the polynucleotides according to (a) or (b).

Applications of the methods according to the invention are for example (i) aiding in the diagnosis and/or prognosis of avian necrotic enteritis, (ii) monitoring the progress or reoccurrence of avian necrotic enteritis, or (iii) aiding in the evaluation of treatment efficacy for an avian subject undergoing or contemplating treatment for necrotic enteritis; wherein the presence or absence of one or more of the biomarkers mentioned before in the nucleic acid extraction obtained from the method is determined, and the one or more biomarkers are associated with the diagnosis, progress or reoccurrence, or treatment efficacy, respectively, of necrotic enteritis.

Applications of the invention in particular help to avoid loss in avian performance like weight gain and feed conversion.

### Isolation of Microvesicles

The isolation of microvesicles from a biological sample prior to extraction of nucleic acids is advantageous, because nucleic acid-containing microvesicles produce significantly higher yields of nucleic acid species with higher integrity as compared to the yield/integrity obtained by extracting nucleic acids directly from the fluid sample or excrement without first isolating microvesicles. Further it is also possible to detect nucleic acids which are expressed at low levels. The isolation of microvesicles also allows the exclusion of proteins, lipids, cell debris, cells and other potential contaminants and PCR inhibitors that are naturally found within biological samples.

Isolation of membrane vesicles from biological samples and subsequent extraction of the nucleic acids can be done for example by ultracentrifugation, e.g., spinning at more than 10,000 g for 1-3 hours, followed by removal of the supernatant, washing the pellet, lysing the pellet and purifying the nucleic acids on a column. Alternative methods are ultrafiltration, e.g., using 100 kD filters, polymer precipitation techniques, and/or filtration based on size.

Further alternative methods are differential centrifugation as described by Raposo et al (1996), Skog et. al (2008) and Nilsson et. al (2009); ion exchange and/or gel permeation chromatography as described in U.S. Pat. Nos. 6,899,863 and 6,812,023; sucrose density gradients or organelle electrophoresis as described in U.S. Pat. No. 7,198,923; magnetic activated cell sorting (MACS) as described by Taylor and Gercel-Taylor (2008); nanomembrane ultrafiltration concentration as described by Cheruvanky et al (2007); Percoll gradient isolation as described by Miranda et al (2010); and, isolation by a microfluidic device Chen et al (2010).

### Capture Surface Based Isolation of Microvesicles According to WO 2014/107571

In a preferred embodiment the microvesicle fraction is bound to a capture surface, in particular to a membrane, a filter or to a surface modified bead as disclosed in WO 2014/107571.

The biological sample is preferably dissolved in a loading buffer, which has preferably a pH of 4-8, more preferably 5-7. Thus, binding to the capture surface takes preferably place at a pH of 4-8, more preferably 5-7. Preferably, after contacting the biological sample with the capture surface, one or more wash steps are performed.

The loading and wash buffers which can be employed according to the invention can be of high or low ionic strength. Thus, the salt concentration, e.g., NaCl concentration, can be from 0 to 2.4M. Preferably, the buffers include one or more of the following components: Tris, Bis-Tris, Bis-Tris-Propane, Imidazole, Citrate, Methyl Malonic Acid, Acetic Acid, Ethanolamine, Diethanolamine, Triethanolamine (TEA) and Sodium phosphate.

Washing preferably takes place by using wash buffer comprising 250 mM Bis Tris Propane and exhibiting a pH of 6.5-7.0.

By adding detergents to the wash buffer nonspecific binding is suppressed. Detergents suitable for use include, but are not limited to, sodium dodecyl sulfate (SDS), Tween-20, Tween-80, Triton X-100, Nonidet P-40 (NP-40), Brij-35, Brij-58, octyl glucoside, octyl thioglucoside, CHAPS or CHAPSO.

After washing of the capture surface the nucleic acid can be released by lysing the fixed microvesicles. For lysing the fixed microvesicles preferably a phenol-based lysis buffer is used, like QIAzol® lysis reagent (Qiagen, Germany). Purification of the nucleic acid can then be done by chloroform extraction using PLG tubes, followed by ethanol conditioning. The nucleic acid can then be bound to a silica column before washing and eluting.

The lysis of microvesicles and extraction of nucleic acids may also be achieved by using a commercially available Qiagen R easy Plus kit, a commercially available Qiagen miR easy kit or by using phenolchloroform according to standard procedures and techniques known in the art. Such methods may also utilize a nucleic acid-binding column to capture the nucleic acids contained within the microvesicles. Once bound, the nucleic acids can then be eluted using a buffer or solution suitable to disrupt the interaction between the nucleic acids and the binding column.

The nucleic acid extraction methods may also include the use of further agents like an RNase inhibitor such as Superase-In (commercially available from Ambion Inc.) or RNaseINplus (commercially available from Promega Corp.); a protease (which may also function as an RNase inhibitor); DNase; a reducing agent; a decoy substrate such as a synthetic RNA and/or carrier RNA; a soluble receptor that can bind RNase; a small interfering RNA (siRNA); an RNA binding molecule, such as an anti-RNA antibody, a basic protein or a chaperone protein; an RNase denaturing substance, such as a high osmolarity solution, a detergent, or a combination thereof. The RNAse inhibitors as well as the further agents may be added to the biological sample, and/or to the isolated microvesicle fraction, prior to extracting nucleic acids.

These further agents may exert their functions in various ways, e.g., through inhibiting RNase activity (e.g., RNase inhibitors), through a ubiquitous degradation of proteins (e.g., proteases), or through a chaperone protein (e.g., a RNA-binding protein) that binds and protects RNAs. In all instances, such extraction enhancement agents remove or at least mitigate some or all of the adverse factors in the biological sample or associated with the isolated particles that would otherwise prevent or interfere with the high quality extraction of nucleic acids from the isolated particles.

The quantification of 18S and 28S rRNAs extracted can be used to determine the quality of the nucleic acid extraction.

The capture surface can be neutral, negatively charged or a positively charged. That means, in principle any kind of capture surface can be used. Depending on the membrane material, the pore sizes of the membrane can range from 20 nm to 5 μm.

In particular the capture surface can be a commercially available membrane like Mustang® Ion Exchange Membrane from PALL Corporation; Vivapure® Q, Vivapure® Q Maxi H; Sartobind® D, Sartobind (S), Sartobind® Q, Sartobind® IDA or Sartobind® Aldehyde from Sartorius AG; Whatman® DE81 from Sigma; Fast Trap Virus Purification column from EMD Millipore; or Thermo Scientific* Pierce Strong Cation and Anion Exchange Spin Columns.

When a membrane is used for fixing the nucleic acids, then the membrane can be made from a variety of suitable materials, for example it can be a polyethersulfone (PES) (e.g., from Millipore or PALL Corp.) or regenerated cellulose (RC) (e.g., from Sartorius or Pierce).

When a negatively charged membrane is used, then the capture surface is preferably an S membrane, which is a negatively charged membrane and is a cation exchanger with sulfonic acid groups. Preferably, the S membrane is functionalized with sulfonic acid, R—CH₂—SO₃″. Alternatively, the negatively charged capture surface is a D membrane, which is a weak basic anion exchanger with diethylamine groups, R—CH₂—NFT(C₂H₅)₂. Alternatively, the capture surface is a metal chelate membrane. For example, the membrane is an IDA membrane, functionalized with minodiacetic acid —N(CH₂COOH″)₂. In some embodiments, the capture surface is a microporous membrane, functionalized with aldehyde groups, —CHO. In other embodiments, the membrane is a weak basic anion exchanger, with diethylamino ethyl (DEAE) cellulose.

When a neutral capture surface is used, then the capture surface is preferably a filter selected from the group consisting of 20 nm neutral PES syringe filtration (Tisch and Exomir), 30 nm neutral PES syringe filtration (Sterlitech), 50 nm neutral PES syringe filtration (Sterlitech), 0.2 um neutral PES homemade spin column filtration (Pall), 0.8 um neutral PES homemade spin column filtration (Pall) and 0.8 um neutral PES syringe filtration (Pall). In embodiments where the capture surface is neutral, preferably the neutral capture surface is not housed in a syringe filter.

The charged surface is preferably selected from the group consisting of 0.65 um positively charged Q PES vacuum filtration, 3-5 um positively charged Q RC spin column filtration, 0.8 um positively charged Q PES homemade spin column filtration, 0.8 um positively charged Q PES syringe filtration, 0.8 um negatively charged S PES homemade spin column filtration, 0.8 um negatively charged S PES syringe filtration, and 50 nm negatively charged nylon syringe filtration.

In the preferred embodiment, when the surface is a positively charged membrane, a Q membrane, which is a positively charged membrane and is an anion exchanger with quaternary amines, can be used. In a preferred embodiment the Q membrane is functionalized with quaternary ammonium, R—CH₂—N⁺(CH₃)₃.

In a very preferred embodiment the positively charged membrane is a regenerated cellulose, strong basic anion exchanger (“RC/SBAE”) membrane, which is an anion exchanger with quaternary amines. In a preferred embodiment the RC/SBAE membrane is functionalized with quaternary ammonium, R—CH₂−N⁺(CH₃)₃. The pore size of the membrane is preferably at least 3 μm.

The capture surface, e.g., membrane, can be housed within a device used for centrifugation; e.g. spin columns, or for vacuum application e.g. vacuum filter holders, or for filtration with pressure e.g. syringe filters.

In a spin-column based purification process preferably more than 1 membrane, preferably 2 to 5 membranes, are used in parallel in a tube, wherein the membranes are preferably all directly adjacent to one another at one end of the column. The collection tube may be commercially available, like a 50 ml Falcon tube. The column is preferably suitable for spinning, i.e., the size is compatible with standard centrifuge and micro-centrifuge machines.

Instead of a membrane also a bead with the same surface modification can be used for capturing the nucleic acid. If a bead is used, it can be magnetic or non-magnetic.

If blood is used as a sample, blood can for example be collected into K2 EDTA tubes and mixed well with complete inversions. Then the tubes are centrifuged at 1300×g for 10 min to separate the blood cells and plasma. Then plasma is removed and filtered through a 0.8 μm filter to remove cell debris and platelets. All plasma samples are then aliquoted into 1 ml cryovials and stored at −80° C. until use.

Before RNA isolation, the membrane is preferably conditioned by passing through equilibrium buffer. The thawed plasma sample is diluted with loading buffer. The diluted plasma sample is slowly passed through the membrane that adsorbs the microvesicles. The membrane is then washed with a wash buffer to remove any weakly bound plasma components. Then a lysis reagent is passed through the membrane to lyse the microvesicles. RNA is isolated using the miRNeasy kit (Qiagen).

RNA can be assessed for quality and concentration with the 2100 Bioanalyzer (Agilent) using a RNA 6000 Pico Chip. The relative quantity of the extracted RNA is measured by RT-qPCR using selected human gene expression assays from Applied Biosystems (Taqman Assay).

In preferred embodiments, in particular when solubilized fecal or cecal samples are used, a pre-processing step prior to isolation, purification or enrichment of the microvesicles is performed to remove large unwanted particles, cells and/or cell debris and other contaminants present in the biological sample. The preprocessing steps may be achieved through one or more centrifugation steps (e.g., differential centrifugation) or one or more filtration steps (e.g., ultrafiltration), or a combination thereof. Where more than one centrifugation pre-processing steps are performed, the biological sample may be centrifuged first at a lower speed and then at a higher speed. If desired, further suitable centrifugation pre-processing steps may be carried out. Alternatively or in addition to the one or more centrifugation pre-processing steps, the biological sample may be filtered. For example, a biological sample may be first centrifuged at a low speed of about 100-500 g, preferably 250-300 g, and after that at a higher speed of about 2,000 to about 200,000 g, preferably at about 20,000 g, for 10 minutes to about 1 hour, preferably at a temperature of 0-10° C., to remove large unwanted particles; the sample can then be filtered, for example, by using a filter with an exclusion size of 0.1 to 1.0 μm.

The microvesicles in the pellet obtained by centrifugation at higher speeds may be reconstituted with a smaller volume of a suitable buffer for the subsequent steps of the process. The concentration step may also be performed by ultrafiltration. In fact, this ultrafiltration both concentrates the biological sample and performs an additional purification of the microvesicle fraction. In another embodiment, the filtration is an ultrafiltration, preferably a tangential ultrafiltration. Tangential ultrafiltration consists of concentrating and fractionating a solution between two compartments (filtrate and retentate), separated by membranes of determined cut-off thresholds. The separation is carried out by applying a flow in the retentate compartment and a transmembrane pressure between this compartment and the filtrate compartment. Different systems may be used to perform the ultrafiltration, such as spiral membranes (Millipore, Amicon), flat membranes or hollow fibers (Amicon, Millipore, Sartorius, Pall, GF, Sepracor). Within the scope of the invention, the use of membranes with a cut-off threshold below 1000 kDa, preferably between 100 kDa and 1000 kDa, or even more preferably between 100 kDa and 600 kDa, is advantageous.

As additional or alternative preprocessing steps size-exclusion chromatography, gel permeation chromatography or affinity chromatography might be carried out before or after contacting the biological sample with the capture surface.

To perform the gel permeation chromatography step, a support selected from silica, acrylamide, agarose, dextran, ethylene glycol-methacrylate copolymer or mixtures thereof, e.g., agarose-dextran mixtures, are preferably used. For example, such supports include, but are not limited to: SUPERDEX® 200HR (Pharmacia), TSK G6000 (TosoHaas) or SEPHACRYL® S (Pharmacia).

Affinity chromatography can be accomplished, for example, by using different supports, resins, beads, antibodies, aptamers, aptamer analogs, molecularly imprinted polymers, or other molecules known in the art that specifically target desired surface molecules on microvesicles.

Optionally, control particles may be added to the sample prior to microvesicle isolation or nucleic acid extraction to serve as an internal control to evaluate the efficiency or quality of microvesicle purification and/or nucleic acid extraction. These control particles include Q-beta bacteriophage, virus particles, or any other particle that contains control nucleic acids (e.g., at least one control target gene) that may be naturally occurring or engineered by recombinant DNA techniques. The control target gene can be quantified using real-time PCR analysis.

Preferably, the control particle is a Q-beta bacteriophage, referred to herein as “Q-beta particle.” The Q-beta particle may be a naturally-occurring virus particle or may be a recombinant or engineered virus, in which at least one component of the virus particle (e.g., a portion of the genome or coat protein) is synthesized by recombinant DNA or molecular biology techniques known in the art. Q-beta is a member of the leviviridae family, characterized by a linear, single-stranded RNA genome that consists of 3 genes encoding four viral proteins: a coat protein, a maturation protein, a lysis protein, and RNA replicase. Due to its similar size to average microvesicles, Q-beta can be easily purified from a biological sample using the same purification methods used to isolate microvesicles, as described herein. In addition, the low complexity of the Q-beta viral single-stranded gene structure is advantageous for its use as a control in amplification-based nucleic acid assays. The Q-beta particle contains a control target gene or control target sequence to be detected or measured for the quantification of the amount of Q-beta particle in a sample. For example, the control target gene is the Q-beta coat protein gene. After addition of the Q-beta particles to the biological sample, the nucleic acids from the Q-beta particle are extracted along with the nucleic acids from the biological sample using the extraction methods and/or kits described herein. Detection of the Q-beta control target gene can be determined by RT-PCR analysis, for example, simultaneously with the biomarkers of interest (i.e., BRAF). A standard curve of at least 2, 3, or 4 known concentrations in 10-fold dilution of a control target gene can be used to determine copy number. The copy number detected and the quantity of Q-beta particle added can be compared to determine the quality of the isolation and/or extraction process.

Thus, 50, 100, 150, 200, 250, 300, 350, 400, 450, 500, 1,000 or 5,000 copies of Q-beta particles added to a bodily fluid sample. In a preferred embodiment, 100 copies of Q-beta particles are added to a bodily fluid sample. The copy number of Q-beta particles can be calculated based on the ability of the Q-beta bacteriophage to infect target cells. Thus, the copy number of Q-beta particles is correlated to the colony forming units of the Q-beta bacteriophage.

### Detection of Nucleic Acid Biomarkers

For detection of RNA, the RNA is preferably reverse-transcribed into complementary DNA (cDNA) before further amplification. Such reverse transcription may be performed alone or in combination with an amplification step. One example of a method combining reverse transcription and amplification steps is reverse transcription polymerase chain reaction (RT-PCT), which may be further modified to be quantitative, e.g. quantitative RT-PCT as described in U.S. Pat. No. 5,639,606. Another example of the method comprises two separate steps: a first of reverse transcription to convert RNA into cDNA and a second step of quantifying the amount of cDNA using quantitative PCR.

RT-PCR analysis determines a Ct (cycle threshold) value for each reaction. In RT-PCR, a positive reaction is detected by accumulation of a fluorescence signal. The Ct value is defined as the number of cycles required for the fluorescent signal to cross the threshold (i.e., exceeds background level). Ct levels are inversely proportional to the amount of target nucleic acid, or control nucleic acid, in the sample (i.e., the lower the Ct level, the greater the amount of control nucleic acid in the sample). Alternatively, the copy number of the control nucleic acid can be measured using another art-recognized techniques.

The analysis of nucleic acids present in the isolated particles is quantitative and/or qualitative. For quantitative analysis, the amounts (expression levels), either relative or absolute, of specific nucleic acids of interest within the isolated particles are measured with methods known in the art. For qualitative analysis, the species of specific nucleic acids of interest within the isolated microvesicles are identified with methods known in the art.

### WORKING EXAMPLES

### Inoculation Preparation

Clostridium perfringens isolate being positive for netB and tpeL was removed from −80° C. storage and aseptically streaked on trypticase soy agar plates containing 5% sheep blood (TSA II). Plates were incubated at 37° C. overnight under anaerobic conditions. Fifteen hours prior to the start of the inoculum culture, an isolated colony from the overnight plate was inoculated into 50 ml pre-reduced BHI broth and incubated at 37° C. under anaerobic conditions for about 15 hours. Then 50 μl of the culture was added to 2×100 ml of pre-reduced BHI broth. The resulting broth-cultures were staggered so that the high dose and low dose reached their final concentrations at the same time (109 cfu/ml and 106 cfu/ml respectively). Aliquots of each culture were taken at the end of the growth period and serially plated onto TSA II agar+5% sheep's blood to verify bacterial concentration.

### Animal Experiment

Seven hundred and twenty (720) male broilers raised in 36 battery pens for 30 days. Birds were equally and randomly assigned (9 pens each) into one of four treatment groups (negative control, sham, low dose gavage and high dose gavage). The birds were fed with standard corn/soy feed without antimicrobials. On the 13th day of rearing, over or under-weight birds were removed from all pens and excluded from the experiment. The bird with normal weight range were then challenged orally with 1 ml of C. perfringens strain harbouring the netB plasmid and being positive for the TpeI gene succesively at days 14, 15 and 16. The sham group was challenged with Iml of PBS, whereas the negative control group was not subjected to any stress or pathogen challenge. Before the first inoculation (day 14), 3 birds from each pen were randomly selected, sacrificed for the collection of blood and digesta samples. Twelve hours after the second challenge, on day 15, 3 birds were again sacrificed from each pen for sample blood and digesta. The gut contents of all sacrificed birds were cultured overnight to determine the ratio of sporulated C. perfringens to the vegetative cells as previously described (Heikinheimo et al, 2004). To assess whether challenge was successful, PCR of NetB and TpeL genes of the gut contents was performed. At least 10 birds were left in each of the pens until the end of the trial. Excreta samples were collected at intervals until study was terminated on day 20 when all birds were sacrificed and three pooled blood samples were obtained from each group. GITs of birds were examined for NE typical signs/lesions and necropsies of the gastro-intestinal tract were taken from duodenum and jejunum for histopathological analysis. Broiler performance indicators like mortality rate, feed consumption and group pathology were determined (table 3).

### Sample Collection

### 1. Fecal and Cecal Excreta Sample Collections

Sheets of paper were placed in the pens to collect pooled fecal and pooled cecal samples every 2 hours after the first challenge until 12 hours after the second inoculation (day 15). Subsequently, samples were collected every 4 hours until day 17. At the end of the trial on day 20, fecal and cecal samples were also collected. During any of the collection event at least 1 gram of fecal and cecal was obtained per pen, and samples were store at −80° C.

### 2. Blood Collection

Blood was collected directly from the heart using a 18 gauge needle. About 5-6 ml of whole blood was collected from 3 randomly selected birds per pen and transferred to collection tubes containing EDTA. Cells are removed from plasma by centrifugation for 10 minutes at 1,000-2,000×g using a refrigerated centrifuge. Following centrifugation, the liquid component (plasma) was immediately transferred into a clean polypropylene tube and stored at −20° C.

3. Gut contents collection

Gut content was collected from a gut approximately 20 cm in length. Gut content was expelled and excised into PBS+20% glycerol and immediately frozen at −20° C. Samples have then been used for quantitation of vegetative versus sporulated C. perfringens and PCR detection of netB and tpeL.

### Histology of Chicken's Intestine

1. Tissue Collection and Processing

A 5 cm section following the duodenal loop was removed from each bird and placed in 10% formalin, and the container was gently agitated to dislodge feed to allow formalin getting into the gut. After fixation, tissues were washed well in several changes of Phosphate Buffered Saline (PBS) and placed in 70% ethanol for storage. For the processing of tissues and infiltration of tissues with paraffin before making 5 uM section, tissues were dehydrated by washing 1× for 2 min in 70% and 95% ethanol solutions successively, and then 2× for 2 mins in 100% ethanol solution.

Finally, tissues were rinsed 2× for 1 minute in Xylene before being embedded in paraffin. The processed tissue was then oriented in a mold, embedded in a paraffin block and cut in a microtome to generate sections of 5 μM. Tissue slices are floated onto Poly-L-lysine (PLL) modified slides. The slides are dried and placed in an oven to “bake” the paraffin.

2. Staining of Tissue Sections with Harris Hematoxylin and Eosin.

In brief, sections were rehydrated by successively decreasing concentration of ethanol: 2× for 3 min each in 100% ethanol, 2× for 3 mins each in 95% ethanol and 1× for 3 minutes in 70% ethanol. Slides were then rinsed for 5 minutes in distilled water and then stained for 6 minutes with Hematoxylin solution (Harris Hematoxylin, Sigma, HHS-32). Slides were rinsed in running tap water for about 20 minutes and decolorized in acid alcohol (95% Ethanol, 2578 ml dH₂O, 950 ml HCL, 9 ml) for 1-3 seconds. Slides were rinsed again with running tap water for 5 minutes then immersed in lithium carbonate for 3 seconds and then rinsed with tap water for 5 minutes. Tissues were counterstained with eosin working solution (100 ml stock eosin (1% aqueous Eosin-Y and 1% aqueous Phloxin B), 10 ml stock Phloxin B, 780 ml 95% Ethanol, 4 ml glacial Acetic Acid) for 15 seconds and rehydrated by successive incubation in ethanol solutions (2 for 3 minutes each in 95% ethanol solution and 2× for 3 minutes each in 100% ethanol solution). Finally, tissues were incubated in xylene solution for 5 minutes and then mounted with cytoseal for analysis.

Detection of Clostridium perfingens netB and tpeL Genes by PCR

TABLE 1
primer pairs used for the detection of netB and
tpeL genes in Clostridium perfringens isolates
from chicken′s intestine

| Primer | | Target | Product |
|---|---|---|---|
| ID | Sequence | Gene | Length |
| netB5F | CGCTTCACATAAAGGTTGGAAGGC | netB | 316 |
| netB5R | TCCAGCACCAGCAGTTTTTCCT | netB | 316 |
| AKP80F | ATATAGAGTCAAGCAGTGGAG | tpeL | 466 |
| AKP81R | GGAATACCACTTGATATACCTG | tpeL | 466 |

### 1. DNA Extraction and Quantification of DNA Concentration

DNA extracted from C. perfringens strains isolated from intestinal contents was performed with commercial DNA extraction kits from Qiagen, Hilden, Germany, according to the manufacturer's instruction.

### 2. DNA Amplification

PCR amplification was performed in a final volume of 25 μl containing 1 ng of DNA template, 0.2 μM of each of the primers for the gene to be amplified or detected and 12 μl of 1×PCR master mix consisting of 1.2 units/25 μL of Taq DNA polymerase. The amplification was carried out in a Gene amplification PCR system, 9600 Thermocycler (Master cycler eppendorf, Germany). Initial denaturation was at 95° C. for 5 min., followed by 40 cycles for 95° C. for 30 sec., annealing at 55° C. for 30 sec and extension at 72° C. for 30 sec, with a final extension at 72° C. for 7 minutes and hold for 40° C.

### Sample Processing

A total of 36 plasma samples, 96 fecal samples and 87 cecal samples were processed for isolation and extraction of microvesicle total RNA. Plasma volumes varied between 0.5 ml to 3 ml from the different animals.

### Plasma Preparation

Briefly, blood was collected into K2 EDTA tubes and mixed well with complete inversions. Then the tubes were centrifuged at 1300×g for 10 min to separate the blood cells and plasma. Then plasma was removed and filtered through a 0.8 μm filter to remove cell debris and platelets. All plasma samples were then aliquoted into Iml cryovials and stored at −80° C. until use.

### Isolation of Microvesicles and RNA Extraction

State of the art methods of extracting microvesicles and further purification of the contents of microvesicles (MV) from biomaterials such as blood, csf, tissues and cell cultures have been described previously in U.S. Pat. No. 6,899,863B1 (Dhellin et al., 2005), EP 2 495 025 A1, WO 2014107571 A and 20140178885A1. Methods include the use of high speed ultracentrifugation, or the use of low speed centrifugation to separate cell debris followed by size exclusion or affinity binding to specific membranes or filtration (see review by Witwer & Colleagues, 2013). Several Immunological methods of purifying microvesicles have also been described (Thery, et al., 2016; Clayton et al., 2001; Wubbolts et al., 2003). Microvesicles can be purified by the use of matrixes, beads or magnetic particles functionalized with antibodies binding to surface proteins of microvesicles to positively select a desired population or with antibodies binding to surface proteins of other cellular components for their depletion (Kim et al., 2012 and Mathivanan et al., 2010). Rapid methods of isolation of microvisicles available to research include the use of microfluidic devices (2010, Chen, et al., 2010; EP 2740536 A2) and commercial kits (e.g., SeraMir™ (System Biosciences), ExoSpin™ (Cell Guidance), ExoMir™ (Bioo Scientifics), miRCURY™ (Exiqon), requiring little or no specialized laboratory equipment.

Herein, extraction of microvesicles from plasma, fecal and cecal samples was performed by the methods described in WO2014/107571A1 (Enderle, et al., 2014). The method involved the isolation of microvesicles from a biological fluid by capturing them on the surface of a Q functionalized membrane filter (Exo50, Exosome Diagnostics). The RNA content of the captured microvesicles was released by lysis with Qiazol, and then the RNA was purified by chloroform extraction or with commercial RNAesay kit from Qiagen. In brief, the plasma sample was allowed to thaw slowly on ice and diluted 1:1 with PBS or 0.9% NaOH; the plasma solution was centrifuged at 300 g for 10 minutes to remove cells and debris. The supernatant was passed through a 0.8 μm filter and then the flow through was centrifuged at 20,000 g at 4° C. for 30 minutes to remove any remaining cell debris. Exo50 column with affinity membrane that binds microvesicles was pre-conditioned by allowing 5 ml loading buffer (2×) 100 mM Phosphate Buffer, 370 mM NaCl) to run through it. Then, the supernatant containing the microvesicles obtained from the second centrifugation step was spiked 1000 copies of Qbeta particle to examine the extraction and recovery efficiency upon loading onto the preconditioned spin column. The column was spun briefly and the flow-through was discarded. The column was washed twice with 2 ml Wash buffer (250 mM Bis Tris Propane, pH6.5-7 and (IX) 50 mM phosphate buffer, 185 mM NaCl) and after the second wash, the column was spun briefly and the flow-through was discarded. Microvesicles bound to the membrane were lysed to release content by the addition of 5 ml (2.25 ml) of lysis buffer (Qiazol) and spun to collect the lysate. RNA was extracted from the lysate with RNeasy kit, Qiagen, following the manufacture's instruction. The RNA eluted with RNase-free water was pooled for duplicated samples to obtain a total volume of 46 μl per sample. 1 μL RNA was applied on Agilent RNA 6000 Nano Kit to assess the quality and concentration of the RNA, while the remaining RNA was stored at −80° C. for further downstream use (QPCR or Sequencing). The extraction efficiency and quantity of RNA extraction was assessed by comparing the Q-beta control target gene (Q-beta coat protein gene) expression in the extracted RNA by RT-PCR analysis to those of the target genes of interest. A standard curve of at least 2, 3, or 4 known concentrations in 10-fold dilution of a control target gene was used to determine the copy number. The copy number detected and the quantity of Q-beta particle added was compared to determine the quality of the isolation and/or extraction process.

### Fecal & Cecal Samples

Since plasma and cecal samples contain large coarse materials, additional preprocessing steps are applied before performing microvesicle isolation and RNA extraction using the Exo50 kit and associated methods as described above for plasma samples. For instance, a 200 mg of fecal sample was allowed to thaw slowly; then 1 ml of cold PBS or 0.9% sterile NaOH was added to homogenize the fecal/cecal sample followed by vortexing for about 1 minute. The resulting suspension is diluted 1:1 with PBS and then centrifuged at 20,000 g for 30 min at 4° C. to remove large unwanted particles. Supernatant was filtered, through a 0.8 μm mixed cellulose ester filter (e.g., Millipore filter) and the filtrate was stored at 4° C. until further use or processed as described for plasma sample above.

TABLE 2
List of primers and probe used for the qPCR to quantify levels of
expression of targets. The miRNA were purchased from Life Technologies
and in house designed primers and probe were synthesized by IDT
(Integrated DNA Technologies, USA)

| Target | Assay ID/Primers and probes (where applicable) |
|---|---|
| miR-155 | TM:000479 (Life Technologies #4427975) |
| miR-16 | TM:000391 (Life Technologies #4427975) |
| miR-24 | TM:000402 (Life Technologies #4427975) |
| miR-206 | TM:000510 (Life Technologies #4427975) |
| virX (IDT) | Forward: 5′-TCGTATTCACTGTAGGAGAACAAG-3′ |
| | Reverse: 5′-TGAGCTTTTCTTGCTTTTCTGC-3′ |
| | Probe: 5′-/56-FAM/ACGATCCAG/ZEN/ |
| | TTAGATGCCCAGCTTG/3IABkFQ/-3′ |
| NetB (IDT) | Forward: 5′-TGGTGCTGGAATAAATGCTTCA-3′ |
| | Reverse: 5′-ACCGTCCTTAGTCTCAACAA-3′ |
| | Probe: 5′-/56-FAM/TGATGCAAA/ZEN/ |
| | TTTAGCATCATGGGA/3IABkFQ/-3′ |
| SAM (IDT) SybrGreen | Forward: 5′-AAATAAGGAGTTGATAAACATGAAA-3′ |
| assay | Reverse: 5′-TGTTACTGGTGAACAACAAAAAT-3′ |
| colA (IDT) | Forward: 5′-TGAAAGAACACCAGAGGAAAGT-3′ |
| | Reverse: 5′-CCTGCAAAGAACTCTGCTGT-3′ |
| | Probe: 5′-/56-FAM/TGATGCAAA/ZEN/GTGGGGGCAAGGA/ |
| | 3IABkFQ/-3′ |
| CPE0956 (IDT) | Forward: 5′-AATAGGATTTATAGCTGGAT-3′ |
| SybrGreen assay | Reverse: 5′-GTTAGTTTTCCAAGGATAG-3′ |
| scRNA (IDT) SybrGreen | Forward: 5′-TATGTAAGTGGTGTTGAGAG-3′ |
| assay | Reverse: 5′-ATGAAAATGACTGCTTATC-3′ |

### QRT-PCR of Fecal/Cecal Derived RNA

Reverse transcription of the fecal and cecal RNA was performed with the SuperScript VILO cDNA Synthesis Kit following the instructions of the manufacturer and the following modifications: the RT reaction consisted of 11 μl (5×VILO Reaction Mix), 5.5 μl (10× SS Enzyme Mix), 5.5 μl (Nuclease free H20) and 33 μl (RNA). While the RT cycle profile consisted of 4 sequential incubation cycles/holding at 250 for 10 min, 42° for 60 min, 85° for 5 min and a final holding step at 4° C.

QRT-PCR for Plasma and Fecal/Cecal Derived miRNA:

Reverse transcription of the miRNA was performed in triplicate for each sample by using Reverse Transcription of RNA using TaqMan MicroRNA Reverse Transcription Kit with the following RT master mix recipe and cycling profile: RT reaction consisted of 6 μl (RT primer pool), 0.3 μl (dNTPs of 100 mM), 1.5 μl (10× RT buffer), 0.2 μl RNase inhibitor, 3.0 μl MultiScribe RT and 4 μl (4 μl RNA for Fecal and cecal samples, and 2 μl RNA+2 μl TE buffer for plasma samples). While the RT cycle profile consisted of 4 sequential incubation cycles/holdings at 16° for 30 min, 42° for 30 min, 85° for 5 min and a final holding step at 4°. The RT primer pool was derived by 10 microliter of each RT primer was pooled and diluted to 1 ml with TE buffer, making the final concentration of each RT primer 0.05×. Five times (5×) of the customized miRNA assay (miR-16, miR-21, miR-24, miR-155 and miR-206) was added to the respective RT primer pool.

### QPCR of Fecal/Cecal RNA

qPCR was performed for probe-based assays for RNA genes using the Qiagen Rotor-Gene Q, according to manufacturer's instruction. The annealing temperature of virX, colA and netB assays was 57° C., while the annealing temperature for the analysis of 16s rRNA of Clostridium perfringens was 60° C. The master mix reactions for the assays consisted of 5.5 μl (Nuclease free water), 10 μl (TaqMan Fast Universal PCR Master Mix no AmpErase UNG), 0.5 μl forward primer (20 uM), 1.5 μl reverse primer (20 μM), 0.25 μl probe (20 μM), 0.25 μl UNG (Uracil-N-glycosylase), and 2 μl cDNA. For the amplification of 16s RNA, 1 μl each of forward and reverse was used.

Rotor-Gene SYBR Green PCR Kit was performed for small non coding RNA VR-RNA, e45nt and scRNA. The master mix reactions for the assays consisted of 5.75 μl (Nuclease free water), 10 μl (Rotor Gene SYBR 2× Master Mix), 0.5 μl forward primer (20 uM), 1.5 μl reverse primer (20 μM), 0.25 μl probe (20 μM), 0.25 μl UNG (Uracil-N-glycosylase), and 2 μl cDNA. The amplification was performed with the following condition: First holding step at 50° C. for 2 min, followed by another hold at 95° C. for 10 and then 40 cycles consisting of 95° C. for 2 seconds, 600 for 15 secs 72° C. for 20 secs.

### Statistical Analysis

All statistical calculations were performed using the statistics software R. As a result of assay's stoichiometry and overall efficiency of the analytical protocols, replacement of missing values (no signal) were imputed before normalization with 40, which is the highest possible CT value in a qPCR with 40 amplification cycles. Moderate statistics for pairwise comparison of the treatment groups were calculated with the ImFit function in the package limma (limma: linear models for microarray data, Smyth G. K. In: Bioinformatics and Computational Biology Solutions using R and Bioconductor, Springer, New York, pp 397-420, R package version 2.16.5) and the resulting p values were adjusted according to the method of Benjamini and Hochberg, (1995) to obtain the q values (false discovery rate)

### Multivariate Analysis (ANOVA)

The dependency between time, treatment and sample type was qualitatively examined. A formal quantitative assessment of possible associations was performed by running two-way ANOVA and non-parametric Kruskal-Wallis tests. Both methods have been applied independently to miRNA and bacterial RNA data. The two way ANOVA was used to test for two main effects and an interaction. The first main effect was time after treatment (in hours), the second main effect was the type of treatment (control, sham, high dose, low dose). The Kruskal-Wallis test was applied separately to time and treatment, to test for an association of these factors with gene expression. No multi-variable interaction was assessed by this method. Three samples types (cecal, excreta and plasma) were analyzed for 4 miRNAs (miR-16, miR-24, miR-155 and miR-206) and a total of 216 duplicate data was available. Also six bacterial targets were analyzed in two sample types (cecal and excreta) and a total of 108 duplicate data was available. The means of the duplicate data (mean Ct value of each sample) were used for further statistical analyses. Analysis was performed on both raw data and normalized data. MiR-191 was used as normalizer for the microRNA analysis, whereas Ct values of 16S rRNA of C. perfringens were used as normarlizer for the bacterial targets. The anova lineal model (Anova (Im(expression-time+treatment+time*treatment)) generated p values for three factors (time-effect, treatment-effect, interaction) for the normalized and raw data, while the 2 p values (time effect and treatment effect) were generated from the analyses with Kruskal-Wallis test for the normalized and raw data.

### Results:

TABLE 3
Number of dead birds per group after C. perfringens challenge. One bird was dead in each
of the groups: low-dose gavage group, the sham group, and the negative control group. 4 birds
were dead in the group of birds challenged with high dose of Clostridium perfringens.

| | Initial | Birds sacrificed | Birds lost after | AFCR* - | Percent with | No signs of |
|---|---|---|---|---|---|---|
| | group | for bleeding days | C. perfringens | after | typical gross | performance |
| Group | size | 14 & 15 | inoculation | challenge | pathology | loss and disease |
| High-dose | 180 | 54 | 4 | 1.60 | 50.70 | 49.30% |
| gavage | | | | | | |
| Low-dose | 180 | 54 | 1 | 1.60 | 42.12 | 57.88% |
| gavage | | | | | | |
| Sham | 180 | 54 | 1 | 1.57 | 6.06^{b} | 93.94% |
| Negative | 180 | 54 | 1 | 1.56 | 3.13 | 96.87% |
| control | | | | | | |
| P value | | | 0.200 | 0.746 | <0.001 | |

*AFCR (adjusted feed conversion ratio) determined as the amount of feed consumed divided by the birds body weight, corrected for mortalities and birds removed due to weight diversion from weight of birds before challenge

TABLE 4
Markers with significantly increased occurrence
in membrane vesicles of cecal excrements

| Marker | Raw FDR | Norm FDR |
|---|---|---|
| Sam region | 1.21E−09 | 9.24E−02 |
| Swim zinc finger region | 9.52E−09 | 9.78E−02 |
| scRNA | 3.21E−05 | 7.93E−03 |
| Mir206 | 8.55E−05 | 9.13E−04 |
| virX | 2.95E−04 | 5.93E−03 |
| netB | 1.04E−03 | 1.30E−03 |
| VR.RNA | 2.89E−03 | 3.21E−05 |
| colA | 6.11E−03 | 3.73E−04 |

TABLE 5
Markers with significantly increased occurrence
in membrane vesicles of fecal excrements

| Marker | Raw FDR | Norm FDR |
|---|---|---|
| Sam region | 2.63E−11 | 8.18E−03 |
| scRNA | 8.94E−05 | 8.64E−04 |
| netB | 2.45E−04 | 2.75E−03 |
| virX | 3.24E−03 | 9.78E−02 |
| colA | 5.01E−03 | 8.68E−04 |
| Swim zinc finger region | 5.81E−02 | 2.26E−03 |
| VR.RNA | 1.36E−01 | 1.03E−04 |

TABLE 6
Markers with significantly increased occurrence
in membrane vesicles of blood samples

| Marker | Norm FDR |
|---|---|
| Mir16 | 6.98E−03 |
| Mir155 | 1.56E−02 |
| Mir24 | 9.40E−02 |

### LIST OF REFERENCES

1. Thery C, Amigorena S, Raposo G, Clayton A (2006). Isolation and characterization of exosomes from cell culture supernatants and biological fluids. Curr Protoc Cell Biol., Chapter 3: Unit 3.22.
2. Clayton A, Court J, Navabi H, Adams M, Mason M D, Hobot J A, et al. (2001). Analysis of antigen presenting cell derived exosomes, based on immuno-magnetic isolation and flow cytometry. J Immunol. Methods. 2001; 247:163-74.
3. Wubbolts R, Leckie R S, Veenhuizen P T, Schwarzmann G, Mobius W, Hoernschemeyer J, et al. (2003). Proteomic and biochemical analyses of human B cell-derived exosomes. Potential implications for their function and multivesicular body formation. J Biol Chem., 278:10963-72.
4. Kim G, Yoo C E, Kim M, Kang H J, Park D, Lee M, et al, (2012) Noble polymeric surface conjugated with zwitterionic moieties and antibodies for the isolation of exosomes from human serum. Bioconjug. Chem., 23:2114-20.
5. Mathivanan S, Lim J W, Tauro B J, Ji H, Moritz R L, Simpson R J (2010). Proteomics analysis of A33 immunoaffinity-purified exosomes released from the human colon tumor cell line LIM1215 reveals a tissue-specific protein signature. Mol. Cell. Proteomics, 9: 197-208.
6. Smyth G. K (2004) linear models for microarray data In: Bioinformatics and Computational Biology Solutions using R and Bioconductor, Springer, New York, pp 397-420
7. Hochberg and Roy (1995) Controlling the false discovery rate: a practical and powerful approach to multiple testing. Journal of the Royal Statistical Soc. Ser B (Methodological), 57:289-300
8. Heikinheimo, A, and Lindström, M, and Korkeala, H. (2004) Enumeration and Isolation of cpe-Positive Clostridium perfringens Spores from Feces. Journal of Clinical Microbiology, 42, 9: 3992-3997
9. Dinh, H., et al., Modulation of microRNAs in two genetically disparate chicken lines showing different necrotic enteritis disease susceptibility. Vet. Immunol. Immunopathol. (2014), http://dx.doi.org/10.1016/j.vetimm.2014.02.003
10. Aade, U. P., and Wankhede, H. J, and Kaldate, K. D (2012). Haematological parameters change in Gallus gallus domesticus infected with cestode parasite. International Multidisciplinary Research Journal, 2(4):13-15
11. Chuku L C et al. (2012) A comparison of the haematological and biochemical indices of broiler and red jungle (hamburgh) fowl (gallus gallus domesticus), Discovery Nature, 2012, 1(1), 15-18,
12. Wang et al. (2012) Integrated analysis of microRNA expression and mRNA transcriptome in lungs of avian influenza virus infected broilers BMC Genomics 2012, 13:278
13. Jiang et al. (2014) Membrane vesicles of Clostridium perfringens type A strains induceinnate and adaptive immunity. International Journal of Medical Microbiology 304 (2014) 431-443
14. Obana, et al. (2013) Structural Requirement in Clostridium perfringens Collagenase mRNA 5′Leader Sequence for Translational Induction through Small RNA-mRNA Base Pairing Journal of Bacteriology, Volume 195 Number 12 p. 2937-2946
15. Ohtani et al. (2002). Identification of a novel locus that regulates expression of toxin genes in Clostridium perfringens FEMS Microbiology Letters, 209: 113-118
16. Nakamura et al. (1995) Small cytoplasmic RNA (scRNA) gene from Clostridium perfringens can replace the gene for the Bacillus subtilis scRNA in both growth and sporulation Microbiology (1995), 141: 2965-2975
17. KEYBURN et al. (2010) Association between avian necrotic enteritis and Clostridium perfringens strains expressing NetB toxin Vet. Res. 41:21
18. Shojadoost et al. (2012). The successful experimental induction of necrotic enteritis in chickens by Clostridium perfringens: a critical review Veterinary Research, 43:74
19. Koga et al. (2011) Exosome can prevent RNase from degrading microRNA in feces. J Gastrointest Oncol 2011; 2: 215-222.
20. Saleem, Gulbeena (2013) Identification of biochemical markers for sub-clinical necrotic enteritis in broiler chickens (In Necrotic enteritis, disease induction, predisposing factors and novel biochemical markers in broilers chickens), Chapter 8, PhD thesis, University of Glasgow, UK.
21. Garcia et al. (2009). Experimental infection of commercial layers using a Salmonella enterica serovar Gallinarum strain: Leukogram and serum acute-phase protein concentrations. Revista Brasileira de Ciencia Avicola 11, 263-270.
22. Piercy (1979). Acute phase responses to experimental salmonellosis in calves and colibacillosis in chickens: serum iron and caeruloplasmin. Journal of Comparative Pathology 89, 309-319.
23. Witwer et al. (2013) Standardization of sample collection, isolation and analysis methods in extracellular vesicle research Journal of Extracellular Vesicles, 2: 20360
24. Nilsson, et al. (2009) Prostate cancer-derived urine exosomes: a novel approach to biomarkers for prostate cancer. Br J Cancer, 100:1603-7.
25. Li et al. (2009) Claudin-containing exosomes in the peripheral circulation of women with ovarian cancer. BMC Cancer, 9:244.
26. Shao et al. (2012) Protein typing of circulating microvesicles allows real time monitoring of glioblastoma therapy. Nat Med., 18:1835-40.
27. Taylor and Gercel-Taylor Taylor (2008) MicroRNA signatures of tumor-derived exosomes as diagnostic biomarkers of ovarian cancer Gynecologic Oncology, 110: 13-21
28. Cheruvanky et al. (2007) Rapid isolation of urinary exosomal biomarkers using a nanomembrane ultrafiltration concentrator. Am J Physiol Renal Physiol., 292: F1657-61.
29. Miranda et al. (2010) Nucleic acids within urinary exosomes/microvesicles are potential biomarkers for renal disease. Kidney Int., 78:191-9.
30. Chen et al. (2010) Microfluidic isolation and transcriptome analysis of serum microvesicles. Lab Chip., 10:505-11.
31. Skog et al. (2008). Glioblastoma microvesicles transport RNA and proteins that promote tumour growth and provide diagnostic biomarkers. Nat Cell Biol., 10:1470-6.
32. Moore et al. (2008). Clostridium toxin netB (WO 2008/148166)
33. Dhellin et al. (2005). Method for preparing membrane vesicles (U.S. Pat. No. 6,899,863B1)
34. Lamparski et al. (2004) Method of producing membrane vesicles (U.S. Pat. No. 6,812,023B1)
35. Abrignani et al. (2007) Method for the preparation of purified HCV RNA by exosome separation (U.S. Pat. No. 7,198,923B1)
36. Giorgio (2012) Filter for the removal of micro-vesicles from biological fluids, methods and devices using such a filter (EP 2495025 A1)
37. Enderle et al (2014) methods for isolating microvesicles (WO2014/107571)
38. Kyung-Hee et al. (2014) Composition and kit for diagnosing breast cancer including polynucleotide within vesicle, and method of diagnosing breast cancer using the same (US 20140178885A1)
39. Min Soak and Jong-Myeon (2014) Microfluidic apparatus and method of enriching target material in biological sample by using the same (EP2740536 A2)

1-15. (canceled)

16. A method of detecting an avian bacterial infection, comprising obtaining an avian sample and subsequently determining the presence and/or level of at least one marker indicative of the bacterial infection, wherein the presence and/or an increased level of this at least one marker in comparison to a non-infected control is indicative of said infection.

17. The method of claim 16, wherein said bacterial infection causes avian necrotic enteritis.

18. The method of claim 17, wherein said avian necrotic enteritis is sub-clinical necrotic enteritis.

19. The method of claim 17, wherein the marker is selected from the group consisting of:
a) a polynucleotide having a sequence identity of at least 80% to at least 40 consecutive nucleotides of a sequence selected from the group consisting of: SEQ ID NO:1; SEQ ID NOs:3-5; SEQ ID NOs: 7-13; and SEQ ID NO:16;
b) a polynucleotide complementary to a sequence of paragraph a);
c) a polynucleotide comprising a sequence of paragraph a).

20. The method of claim 17, wherein the marker is selected from the group consisting of:
a) a polynucleotide which has a sequence identity of at least 90% to a polynucleotide which comprises at least 40 consecutive nucleotides of the nucleotide sequence according to positions 420 to 630 of SEQ ID NO: 1;
b) a polynucleotide which has a sequence identity of at least 90% to a polynucleotide which comprises at least 40 consecutive nucleotides of the nucleotide sequence according to positions 670 to 760 of SEQ ID NO:3;
c) a polynucleotide which has a sequence identity of at least 90% to a polynucleotide which comprises at least 40 consecutive nucleotides of the nucleotide sequence according to positions 80 to 220 of SEQ ID NO:4;
d) a polynucleotide which has a sequence identity of at least 90% to a polynucleotide which comprises at least 40 consecutive nucleotides according to positions 1400 to 1700 of SEQ ID NO:5;
e) a polynucleotide which has a sequence identity of at least 90% to a polynucleotide which comprises at least 40 consecutive nucleotides of the nucleotide sequence according to positions 100 to 280 of SEQ ID NO:7;
f) a polynucleotide which has a sequence identity of at least 90% to a polynucleotide which comprises at least 40 consecutive nucleotides of the nucleotide sequence according to positions 300 to 540 of SEQ ID NO:8;
g) a polynucleotide which have a sequence identity of at least 90% to a polynucleotide which comprises at least 40 consecutive nucleotides of the nucleotide sequence according to positions 240 to 400 of SEQ ID NO:9;
h) a polynucleotide which has a sequence identity of at least 90% to a polynucleotide which comprises at least 40 consecutive nucleotides of the nucleotide sequence according to positions 673 to 5628 of SEQ ID NO: 16;
i) a polynucleotide which has a sequence identity of at least 90%, to a polynucleotide which comprises at least 12 consecutive nucleotides of the nucleotide sequence according to position 14 to 25 of SEQ ID NO: 10;
j) a polynucleotide which has a sequence identity of at least 90% to a polynucleotide which comprises at least 12 consecutive nucleotides of the nucleotide sequence according to position 44 to 65 of SEQ ID NO: 11;
k) a polynucleotide which has a sequence identity of at least 90% to a polynucleotide which comprises at least 12 consecutive nucleotides of the nucleotide sequence according to position 3 to 24 of SEQ ID NO: 12;
l) a polynucleotide which has a sequence identity of at least 90% to a polynucleotide which comprises at least 12 consecutive nucleotides of the nucleotide sequence according to position 45 to 66 of SEQ ID NO: 13;
m) a polynucleotide which is complementary to a sequence according to paragraphs (a) to (l);
n) a polynucleotide which comprises a sequence according to paragraphs (a) to (m).

21. The method of claim 17, wherein the marker is selected from the group consisting of:
a) a polynucleotide having a sequence identity of at least 90% to at least 12 consecutive nucleotides of a sequence selected from the group consisting of SEQ ID NOs:10-13;
b) a polynucleotide complementary to a sequence of paragraph a);
c) a polynucleotides comprising a sequence of paragraph a).

22. The method of claim 16, wherein said method comprises isolating microvesicles from an avian sample and subsequently determining the presence and/or level of at least one avian marker indicative for the infection, wherein the presence and/or an increased level of this at least one avian marker in comparison to a non-infected control is indicative for the infection and wherein the at least one avian marker is an avian miRNA and is selected from the group consisting of:
a) a polynucleotide having a sequence identity of at least 90% to at least 12 consecutive nucleotides of a sequence selected from the group consisting of SEQ ID NOs:10-13;
b) a polynucleotide complementary to the sequences of paragraph a);
c) a polynucleotide comprising the sequences of paragraph a).

23. The method of claim 16, wherein the avian sample is a bodily fluid.

24. The method of claim 17, wherein said method comprises determining the presence and/or level of at least one marker indicative of necrotic enteritis in avian fecal excrements, wherein the marker is selected from the group consisting of:
a) a polynucleotide having a sequence identity of at least 80% to at least 40 consecutive nucleotides of a sequence selected from the group consisting of: SEQ ID NO:1; SEQ ID NOs:3-5; and SEQ ID NOs:7-9;
b) polynucleotides complementary to the sequences of paragraph a);
c) polynucleotides comprising the sequences of paragraph a).

25. The method of claim 17, wherein said method comprises determining the presence and/or level of at least one marker indicative of necrotic enteritis in avian blood wherein the marker is selected from the group consisting of:
a) a polynucleotide having a sequence identity of at least 90% to at least 12 consecutive nucleotides of a sequence selected from the group consisting of: SEQ ID NOs:10-12;
b) polynucleotides complementary to the sequences of paragraph a);
c) polynucleotides comprising the sequences of paragraph a).

26. The method of claim 16, wherein said method comprises determining the presence and/or amount of at least one marker indicative of the disease in microvesicles isolated from avian excrements.

27. The method of claim 16, wherein the avian sample is obtained from poultry,

28. The method of claim 16, wherein the avian sample is obtained from an avian animal selected from the group consisting of: chickens, turkeys, ducks, geese, peacocks, pheasants, partridges, guinea fowl, quails, capercaillies, grouse, pigeons, swans, ostriches and parrots, and chickens.

29. A polynucleotide selected from the group consisting of:
a) a polynucleotide which has a sequence identity of at least 80% to a polynucleotide which comprises at least 100 consecutive nucleotides of a sequence selected from the group consisting of SEQ ID NO:1; SEQ ID NO:3 and SEQ ID NO:7;
b) a polynucleotide complementary to a sequence of paragraph a);
c) a polynucleotide comprising the sequence of paragraph a).

30. The polynucleotide of claim 29, wherein said polynucleotide has a sequence identity of at least 90% to a polynucleotide which comprises at least 100 consecutive nucleotides of a sequence selected from the group consisting of SEQ ID NO:1; SEQ ID NO:3 and SEQ ID NO:7.

31. A polypeptide selected from the group consisting of:
a) a polypeptide which has a sequence identity of at least 80% to a polypeptide which comprises at least 15 consecutive amino acids to a sequence selected from the group consisting of: SEQ ID NO:2; SEQ ID NO:14; and SEQ ID NO:15;
b) a polypeptide which comprises the polypeptides of paragraph a).
